(19) United States
(12) Patent Application Publication
(10) Pub. No.: US 2024/0182552 A1
(43) Pub. Date: Jun. 6, 2024

(54) PHARMACEUTICAL COMPOSITION FOR PREVENTING OR TREATING IMMUNE DYSREGULATION-RELATED DISEASES COMPRISING OXIDIZED IMMUNOGLOBULIN AS ACTIVE INGREDIENT

(71) Applicant: AJOU UNIVERSITY INDUSTRY-ACADEMIC COOPERATION FOUNDATION, Suwon-si, Gyeonggi-do (KR)

(72) Inventor: Dong Ho Nahm, Seongnam-si, Gyeonggi-do (KR)

(73) Assignee: AJOU UNIVERSITY INDUSTRY-ACADEMIC COOPERATION FOUNDATION, Suwon-si, Gyeonggi-do (KR)

(21) Appl. No.: 18/285,862

(22) PCT Filed: Apr. 6, 2022
(86) PCT No.: PCT/KR2022/004917

§ 371 Date: Oct. 6, 2023

(30) Foreign Application Priority Data
Apr. 7, 2021 (KR) 10-2021-0045451
Apr. 5, 2022 (KR) 10-2022-0042074

(51) Int. Cl. C07K 16/18; A61K 39/00; A61P 35/00; A61P 37/08; C07K 16/06

(52) U.S. Cl. CPC C07K 16/18; A61P 35/00; A61P 37/08; C07K 16/065; A61K 2039/505

(57) ABSTRACT

The present invention relates to a pharmaceutical composition for preventing or treating immune dysregulation-related diseases, including oxidized immunoglobulin as an active ingredient and a preventive or therapeutic method using same, a pharmaceutical composition for treating allergic diseases, chronic inflammatory diseases, autoimmune diseases or malignant tumor diseases, including as an active ingredient oxidized immunoglobulin obtained through artificially induced oxidation; and a novel immunomodulatory therapy which, by administering the composition to patients with the diseases, is differentiated from existing immunoglobulin therapeutic agents and more effective.

### TECHNICAL FIELD

The present disclosure relates to a pharmaceutical composition for preventing or treating diseases associated with immune dysregulation including oxidized immunoglobulin as an active ingredient, and a treatment method using the same.

### BACKGROUND ART

It is well known that immune dysregulation, including hypersensitive immune response (allergic reaction), excessive immune response to one's own antigen (autoimmune response), or low immune response to tumor cells (in malignant tumor disease), plays a key role in the pathogenesis of allergic diseases, chronic inflammatory diseases or autoimmune diseases, and malignant tumor diseases. Accordingly, an immunomodulatory therapeutic effect of various types of immunoglobulin therapeutic agents, including monoclonal antibody therapeutic agents, has been identified in patients suffering from the diseases associated with immune dysregulation, and thus the therapeutic agents are actually used in clinical practice.

Immunoglobulin therapeutic agents that are currently used for prevention or treatment of allergic diseases, chronic inflammatory diseases or autoimmune diseases, and malignant tumor diseases are produced in two forms which are monoclonal antibodies that specifically respond to specific target antigens involved in the immune response or polyvalent or polyclonal immunoglobulin preparations isolated from plasma pools obtained from a large number of healthy blood donors, so as to be used for prevention or treatment of various diseases associated with immune dysregulation occurring in humans or mammals. However, the mechanism how the polyvalent or polyclonal immunoglobulin preparations isolated from the plasma pool from a large number of healthy blood donors exhibit immunomodulatory therapeutic effects in the diseases has not been clearly revealed.

Currently, immunoglobulin preparations that are commonly used show preventive and therapeutic effects in the diseases when administered mostly subcutaneously, intramuscularly, or intravenously. In the case of monoclonal antibodies against specific proteins involved in immune responses, when injected into mammals suffering from diseases, they are known to exhibit immunomodulatory effects by specifically inhibiting activation of specific immune pathways in which the protein is involved. Some researchers have proposed hypothesis (hypothesis of passive anti-idiotype therapy) on the mechanism of the polyvalent immunoglobulin preparations in alleviating diseases, wherein the polyvalent immunoglobulin preparation isolated from the blood of a large number of healthy donors include naturally produced anti-idiotype antibodies that react to antigen binding sites (idiotypes) of pathogenic antibodies contributing to development of diseases, such that when administered to patients, the naturally produced anti-idiotype antibodies react with immunoglobulin E (IgE) against allergens or immunoglobulin G (IgG) against autoantigens (which are pathological antibodies that play a crucial role in development of diseases) and form antigen-antibody immune complexes, thereby inhibit functions of pathological antibodies.

Unlike the existing hypothesis on the passive anti-idiotype therapy set forth above, in order to induce active anti-idiotype immunotherapeutic effects using immunoglobulin proteins as an antigen, the present inventor designed the present disclosure by setting a hypothesis (hypothesis of active anti-idiotype therapy) that artificial oxidation of immunoglobulin proteins can increase immunogenicity of immunoglobulins and efficiently produce an effect of the active anti-idiotype therapy. Currently, the industry is striving to block oxidation of the immunoglobulin protein in the production process of immunoglobulin therapeutic agents (especially monoclonal antibody therapeutic agents) as much as possible, because (1) it (oxidation of the immunoglobulin) can inhibit an action of therapeutic antibodies by increasing production of anti-idiotypic antibodies (i.e., anti-drug antibodies) against antigen binding sites of the immunoglobulin used as a therapeutic agent when the oxidized immunoglobulin protein is administered to the human body, (2) it (oxication of the immunoglobulin) can induce protein aggregation by formation of polymers and increases the protein loss, and (3) it (oxication of the immunoglobulin) can increase the risk of side effects of inflammatory reactions due to complement activation in patients administered with aggregated immunoglobulins.

Aside from a passive immunotherapy to induce an effect of blocking a specific immune response pathway using monoclonal antibodies or to induce an effect of blocking multiple pathological antibodies using polyvalent immunoglobulins targeted by immunoglobulin preparations that are currently used for treatment of diseases associated with immune dysregulation in humans, the inventor, through this invention, has attempted to induce active immune responses against antigen binding sites (idiotype) of immunoglobulins (active anti-idiotype immunotherapy) by using immunoglobulin itself as an antigen to be administered to mammals. Accordingly, the inventor proposed a new pharmaceutical composition for immunomodulatory therapies and a new treatment method using the same, capable of preventing or treating diseases associated with immune dysregulation using oxidized immunoglobulins more effectively than conventional immunoglobulin therapeutic agents in this invention.

### DISCLOSURE OF THE INVENTION

### Technical Goals

The present disclosure provides a new pharmaceutical composition for immunomodulatory therapies that exhibits a better immunomodulatory effect than previously used immunoglobulins when administered to patients suffering from diseases associated with immune dysregulation, and a method of preventing or treating diseases associated with immune dysregulation using the pharmaceutical composition.

### Technical Solutions

The present disclosure can provide a pharmaceutical composition for preventing or treating diseases associated with immune dysregulation including oxidized immunoglobulin as an active ingredient.

The oxidized immunoglobulin may be obtained by mixing and reacting immunoglobulin with one or more oxidants from the group consisting of oxidants including ozone (O₃), hydrogen peroxide (H₂O₂), and sodium hypochlorite (NaClO).

The oxidized immunoglobulin may be obtained by mixing and reacting 0.1 to 4 μg of ozone per 1 mg of immunoglobulin.

The immunoglobulin may be any one from the group consisting of IgG, IgA, IgM, IgD, and IgE.

The immunoglobulin may be IgG.

The IgG may be an immunoglobulin G isolated from blood of a mammal.

The IgG may be IgG isolated from the blood of the mammal itself or IgG isolated from the blood of another mammal.

The IgG described above may be IgG isolated from a culture medium of animal cells.

The disease associated with immune dysregulation may be any one from the group consisting of allergic diseases, chronic inflammatory diseases, autoimmune diseases, and malignant tumor diseases.

The allergic disease may be any one from the group consisting of bronchial asthma, allergic rhinitis, allergic conjunctivitis, atopic dermatitis, urticaria, food allergy, anaphylaxis, and drug allergy.

The chronic inflammatory disease or autoimmune disease may be any one from the group consisting of degenerative arthritis, chronic inflammatory gastroenteritis, ankylosing spondylitis, chronic pustular dermatitis, rheumatoid arthritis, systemic lupus erythematosus, pemphigus, autoimmune thyroiditis, autoimmune hepatitis, chronic inflammatory bowel disease, autoimmune nephritis, chronic inflammatory gastroenteritis, Sjögren syndrome, scleroderma, and psoriasis.

The malignant tumor disease may be a solid cancer selected from the group consisting of lung cancer, gastric cancer, colorectal cancer, pancreatic cancer, ovarian cancer, uterine cancer, thyroid cancer, breast cancer, liver cancer, kidney cancer, prostate cancer, and mesothelioma, or any one of hematologic malignancy selected from the group consisting of leukemia, lymphoma, and multiple myeloma.

The oxidized immunoglobulin may have an anticancer immunotherapeutic effect by immunomodulation through activation of cytotoxic T cells.

In addition, the present disclosure provides a novel pharmaceutical composition for immunomodulatory therapies obtained by mixing an immunoadjuvant in addition to the oxidized immunoglobulin, and a preparation method thereof. The immunoadjuvant included in the pharmaceutical composition described above may be aluminum hydroxide, calcium phosphate, tyrosine, monophosphoryl lipid A (MPL), or histamine, that are currently commonly used in humans for the purpose of immunopotentiation in current vaccine formulations or immunomodulatory therapeutic agents.

In addition, the present disclosure may provide a method of preventing or treating diseases associated with immune dysregulation, including isolating immunoglobulin from blood of a mammal itself or isolating immunoglobulin from blood of another mammal (step 1), mixing and reacting the isolated immunoglobulin with an oxidant to prepare oxidized immunoglobulin (step 2), and administering the oxidized immunoglobulin to an individual suffering from a disease associated with immune dysregulation (step 3).

The oxidant described above may be one selected from ozone, hydrogen peroxide, and sodium hypochlorite.

The step 2 may include mixing and reacting 0.1 to 4 μg of ozone per 1 mg of isolated immunoglobulin.

In addition, the present disclosure may provide a method of preparing a pharmaceutical composition for preventing or treating diseases associated with immune dysregulation, including isolating immunoglobulin from blood of a mammal itself or isolating immunoglobulin from blood of another mammal (step 1), and mixing and reacting the isolated immunoglobulin with an oxidant to prepare oxidized immunoglobulin (step 2).

The oxidant described above may be one selected from ozone, hydrogen peroxide, and sodium hypochlorite.

The step 2 may include mixing and reacting 0.1 to 4 μg of ozone per 1 mg of isolated immunoglobulin.

### Advantageous Effects

According to the present disclosure, when a pharmaceutical composition of the present disclosure is administered to patients with diseases associated with immune dysregulation including oxidized immunoglobulins with increased immunogenicity as an active ingredient by artificially inducing oxidation, this can produce significantly enhanced immunomodulatory therapeutic effects compared to a case in which a conventional therapeutic agent including non-oxidized immunoglobulins as an active ingredient is administered, therefore this invention can provide a pharmaceutical composition ensuring excellent preventive and therapeutic effects on diseases associated with immune dysregulation.

### BRIEF DESCRIPTION OF DRAWINGS

FIG. 1 shows a result graph of comparing and analyzing, through Kaplan-Meier analysis in patients with advanced solid tumors accompanied by distant metastases, a survival period and cumulative survival rate of patient group treated by intramuscular injection of autologous total immunoglobulin G (IgG) oxidized by a reaction with ozone (oxidized autologous IgG treatment group) and patient group treated by intramuscular injection of non-oxidized autologous total immunoglobulin G (IgG) (autologous IgG treatment group), and a survival rate at 1 month, 3 months, and 6 months after receiving the treatment described above in the patients participated in this clinical trial.

FIG. 2 shows results of analyzing a change in a proportion (%) of IFN-γ producing cells among peripheral blood CD8+ T cells by flow cytometry at a start of treatment (week 0) and at 4 weeks after completion of treatment (week 8) of a first cycle in two patients with advanced solid tumors who received intramuscular injection of oxidized autologous total IgG.

FIG. 3 shows a result of confirming whether the immunoglobulins are oxidized or not oxidized after carrying out oxidation by mixing commercialized immunoglobulins for intramuscular injection with ozone and sodium hypochlorite by immunoblot method using anti-DNP antibody.

FIG. 4 shows a result of confirming whether the immunoglobulins are oxidized or not after carrying out oxidation process by mixing commercialized human immunoglobulins for intramuscular injection and human immunoglobulins for intravenous injection with ozone.

FIG. 5 shows a design of a clinical trial study conducted in one healthy normal human subject to determine a difference in immunomodulatory effects between a case received a intramuscular injection of commercially available IgG for intramuscular injection prepared by isolating from the blood of multiple healthy donors and a case received a intramuscular injection of IgG underwent oxidation by mixing the IgG with ozone.

FIG. 6 shows a graph of comparing IL-10 concentrations in culture mediums obtained after reacting cultured human peripheral blood mononuclear cells isolated from the peripheral blood of a healthy normal human subject (blood donor 1) with human IgG or oxidized human IgG.

FIG. 7 shows a graph of comparing IL-10 concentrations in culture mediums obtained after reacting cultured human peripheral blood mononuclear cells isolated from the peripheral blood of a healthy normal human subject (blood donor 2) with human IgG or oxidized human IgG.

FIG. 8 shows a graph of analyzing differences in IL-10 concentrations in culture mediums according to a change in a degree of oxidation of human IgG when cultured human peripheral blood mononuclear cells from the peripheral blood of a healthy normal human subject (blood donor 2) are reacted with human IgGs oxidized under various conditions.

FIG. 9 shows a graph of comparing IL-8 concentrations in culture mediums obtained after reacting cultured human peripheral blood mononuclear cells isolated from the peripheral blood of a healthy normal human subject (blood donor 1) with human IgG or oxidized human IgG.

FIG. 10 shows a graph of comparing IL-8 concentrations in culture mediums obtained after reacting cultured human peripheral blood mononuclear cells isolated from the peripheral blood of a healthy normal human subject (blood donor 2) with human IgG or oxidized human IgG.

FIG. 11 shows a graph of comparing differences in IL-8 concentrations in culture mediums according to a change in a degree of oxidation of human IgG when cultured human peripheral blood mononuclear cells isolated from the peripheral blood of a healthy normal human subject (blood donor 2) are reacted with human IgGs oxidized under various conditions.

FIG. 12 shows a graph of comparing ovualbumin (OVA)-specific IgG antibody titers in serum specimens of mice obtained after subcutaneous injection of human IgG or oxidized human IgG in the OVA allergy mouse model.

FIG. 13 shows a graph of comparative analysis of a difference in a β-hexosaminidase secretion release rate (% release) from rat basophilic leukemia cells when by IgE-antigen-mediated stimulation when rat basophilic leukemia cells were pre-treated with human IgG and oxidized human IgG.

### BEST MODE FOR CARRYING OUT THE INVENTION

Hereinafter, the present disclosure will be described in more detail.

In example embodiments of the present disclosure, the present inventors completed the present disclosure by confirming, (1) when autologous immunoglobulins are isolated from the blood of patients suffering from diseases associated with immune dysregulation to artificially subjected to oxidation and then the oxidized autologous immunoglobulins are administered to the patient by intramuscular injection, that significantly excellent disease therapeutic effects were shown compared to the case in which non-oxidized autologous immunoglobulins are injected intramuscularly, (2) the oxidized immunoglobulin induced a significantly improved immunomodulatory effect in normal people and experimental animals compared to conventional non-oxidized immunoglobulins, and, (3) even in immune cells cultured under laboratory conditions, the oxidized immunoglobulins can exhibit significantly enhanced immunomodulatory therapeutic effects compared to conventional non-oxidized immunoglobulins.

The present disclosure relates to a pharmaceutical composition for preventing or treating diseases associated with immune dysregulation including oxidized immunoglobulins as a main active ingredient.

The present disclosure relates to a method of preventing or treating diseases associated with immune dysregulation using oxidized immunoglobulins.

The term “composition” as used in the specification of the present disclosure is considered to include not only a product including a particular component, but also any product directly or indirectly produced by combination of particular components.

Each active ingredient used in the composition of the present disclosure may be present in the composition of the present disclosure, an injection preparation in which the composition of the disclosure is dissolved, or one or more of the vivo, and immunoadjuvants or histamine may be present in the form of a complex covalently or non-covalently bound with immunoglobulins, respectively.

The composition of the present disclosure may include a composition in which one active ingredient is in a pharmaceutically or physiologically acceptable form, a composition in which all active ingredients are in the form of a pharmaceutically or physiologically acceptable salt, a composition in which one or more active ingredients are in the form of a pharmaceutically or physiologically acceptable salt while other active ingredients are in the form of free base, or a composition in which a complex of one or more active ingredients is in the form of a pharmaceutically or physiologically acceptable salt.

The active ingredients may be thoroughly mixed with various forms of pharmaceutically acceptable carriers, depending on the form of the preparation required for administration. The pharmaceutical composition of the present disclosure may preferably be in the form of a unit dose, and it may be possible to use in a diluted form so that the dose may be adjusted to be used according to the judgment of the doctor.

The term “immunoglobulin” as used herein refers to a glycoprotein that plays an important role in immunity among serum components and may be limited to common features such as specific physical, structural amino acid sequences that act as antibodies. The immunoglobulin has a basic structure in which one pair of L chains (light chains) with a molecular weight of about 23,000 and one pair of H chains (heavy chains) with a molecular weight of about 50,000 to 70,000 are conjugated by S—S bonds, and are respectively classified into IgG, IgA, IgM, IgD, and IgE depending on the type of H chains such as γ, α, μ, δ, and ε. The immunoglobulins used in the composition of the present disclosure may be IgG, IgA, IgM, IgD, IgE or mixtures thereof and be fragments thereof or mixtures thereof having biologically equivalent activity.

In the present disclosure, the immunoglobulin may be a total immunoglobulin including IgG, IgA, IgM, IgD, and IgE isolated from the blood of mammals. In detail, the immunoglobulin may be IgG isolated from the blood of mammals suffering from diseases related to immune dysregulation.

The immunoglobulins used in the composition, prevention method, or treatment method of the present disclosure may be isolated, for example, using the following methods. In order to isolate total immunoglobulins from mammalian blood or plasma, isolation may be performed by various methods that are commonly used in the art, including ethanol precipitation, ion exchange resin adsorption chromatography, or affinity chromatography using Protein A or Protein G columns. On the other hand, according to methods known in the art, used may be those prepared by using genetically engineered cultured animal cells on the basis of acquired information on immunoglobulins from a cDNA library having genetic information on antibody proteins obtained from peripheral blood mononuclear cells of mammals. The genetically recombinant immunoglobulin thus prepared may include a genetically engineered recombinant immunoglobulin protein obtained by oxidizing human immunoglobulin with amino acid sequence of the mammalian immunoglobulin or by partially altering the same. In addition, the immunoglobulin may be a part of an immunoglobulin that binds to an allergen, such as an F(ab)′2 or Fab fragment that may react against an allergen among immunoglobulin proteins.

In addition, the immunoglobulin used in the composition of the present disclosure may be obtained from an animal of species different from the mammal to which the composition of the present disclosure is to be administered. In particular, it is already widely known in the art that immunoglobulins have high homology among heterogenous species, so that immunoglobulins obtained from one species of mammals may exhibit similar pharmacological effects even when administered to mammals of other species, including humans. Therefore, the effect of preventing or treating immune dysregulation by administering the composition of the present disclosure may be the same even if the pharmaceutical composition of the present disclosure is finally obtained from a mammal of a different species from the animal administered for the purpose of suppressing the immune dysregulation responses.

The term “mammal” as used herein refers to a mammal that is a subject of treatment, observation, or experiment, preferably a human.

In the present disclosure, the above oxidized immunoglobulin may refer to an antibody, when administered to the mammal: the oxidized immunoglobulin itself is used as an antigen to enable natural production of anti-idiotypic antibodies reacting to antigen binding sites of the pathological immune antibody and can form antigen-antibody immune complexes.

Diseases associated with immune dysregulation in the present disclosure may be, but are not limited to, allergic diseases, chronic inflammatory diseases, autoimmune diseases, or malignant tumor diseases.

The allergic disease may be selected from the group consisting of, but is not limited to, bronchial asthma, allergic rhinitis, allergic conjunctivitis, atopic dermatitis, urticaria, food allergy, anaphylaxis, and drug allergy which are known to be caused by allergic reactions against antigens present in the external environment.

The chronic inflammatory disease or autoimmune disease may be selected from the group consisting of, but is not limited to, degenerative arthritis, chronic inflammatory gastroenteritis, ankylosing spondylitis, chronic pustular dermatitis, rheumatoid arthritis, systemic lupus erythematosus, pemphigus, autoimmune thyroiditis, autoimmune hepatitis, chronic inflammatory bowel disease, autoimmune nephritis, chronic inflammatory gastroenteritis, Sjögren syndrome, scleroderma, and psoriasis.

The malignant tumor disease may be a solid cancer selected from the group consisting of lung cancer, gastric cancer, colorectal cancer, pancreatic cancer, ovarian cancer, uterine cancer, thyroid cancer, breast cancer, liver cancer, kidney cancer, prostate cancer, and mesothelioma, or may be selected from hematologic malignancy selected from the group consisting of leukemia, lymphoma, and multiple myeloma, but is not limited thereto.

The present disclosure provides a novel pharmaceutical composition for immunomodulatory therapies in which an immunoadjuvant is mixed in addition to the oxidized immunoglobulin and a preparation method thereof.

The immunoadjuvant included in the pharmaceutical composition may be aluminum hydroxide, calcium phosphate, tyrosine, monophosphoryl lipid A (MPL), or histamine, that are currently commonly used in humans for the purpose of immunopotentiation in current vaccine formulations or immunomodulatory therapeutic agents.

The present disclosure may provide a pharmaceutical composition for preventing or treating allergic diseases, chronic inflammatory diseases, autoimmune diseases, or malignant tumor diseases, including oxidized immunoglobulins as an active ingredient.

In addition, the present disclosure may provide a method of preventing or treating diseases associated with immune dysregulation, including isolating immunoglobulins from the blood of a mammal itself or other mammals (step 1), mixing and reacting the isolated immunoglobulin with an oxidant to prepare oxidized immunoglobulins (step 2), and administering the oxidized immunoglobulin to an individual suffering from diseases associated with immune dysregulation (step 3).

According to the present disclosure, the oxidized immunoglobulin may be prepared via a reaction by mixing immunoglobulins and the oxidant, and the oxidant may be selected from the group consisting of ozone (O₃), hydrogen peroxide (H₂O₂), and sodium hypochlorite (NaClO) that are known to be able to generate oxygen free radicals or reactive oxygen species, but is not limited thereto.

According to an example embodiment of the present disclosure, the oxidized immunoglobulin may be prepared by carrying out a reaction by mixing immunoglobulins in liquid state with hydrogen peroxide or sodium hypochlorite in liquid state or with ozone gas. The reaction may be carried out at a temperature of 4 to 95° C. for 1 minute to 24 hours.

In this case, the consumption of ozone gas for 1 mg of human IgG (hIgG) may be 0.01 to 10 μg, specifically 0.1 to 4 μg.

The pharmaceutical composition of the present disclosure is preferably for subcutaneous injection. However, in an example embodiment of the present disclosure, the composition may be administered in a conventional manner via intravenous, intraarterial, intramuscular, intraperitoneal, intrasternal, transdermal, intranasal, inhalational, topical, rectal, oral, intraocular, or intradermal routes.

The dose of the pharmaceutical composition of the present disclosure may be determined in consideration of the dose of immunoglobulins used in treatment with immunoglobulin formulations that are currently in use. In the case of a general pharmaceutical composition, the dose of the composition may be determined according to the severity of symptoms, the age of the patient, and the weight, but in the treatment method of the present disclosure, it may be determined according to the patient's sensitivity to oxidized immunoglobulins as well as the above conditions.

In pharmaceutical composition of the present disclosure, a single dose of the oxidized immunoglobulin may be 0.001 to 1000 mg, specifically 10 to 50 mg, but is not limited thereto. The composition may preferably be present in the form of a solution or lyophilized powder and may be used in a form included in 0.5 to 2 ml of injection buffer in a single dose.

In addition, to be used by dissolving in the injection buffer contained in a separate vial, immunoadjuvants, including aluminum hydroxide, calcium phosphate, tyrosine, and monophosphoryl lipid A (MPL) which are commonly used to increase the immune responses with immunoglobulins, or histamine may be provided in a mixed formulation form, and the doctor may determine the dose for use according to the patient's symptoms before administration.

The injection buffer and other additional components used to prepare the composition of the present disclosure into an injection preparation are known in the art. In addition to the injection buffer, the preparation for injection for the composition of the present disclosure may include, for example, other additional components such as dissolution aids, pH adjusters, and suspensions. For example, normal saline may be used for the injection buffer.

It is apparent to those skilled in the art that the therapeutically effective dose and the number of administrations for the active ingredients of the present disclosure or the pharmaceutical compositions including the same will vary depending on the desired effect. Therefore, the optimal dose to be administered may be easily determined and vary depending on the specific active ingredient used, the type of administration, an effect of the preparation, and the development of a disease condition. In addition, dose adjustment according to the appropriate therapeutic level will be required depending on factors of each patient being treated, including the patient's age, weight, diet, and time of administration.

Hereinafter, example embodiments will be described in detail to help the understanding of the present disclosure. However, the following example embodiments are merely illustrative of the content of the present disclosure, and the scope of the present disclosure is not limited to the following example embodiments. The example embodiments of the present disclosure are provided to more completely explain the present disclosure to those skilled in the art.

### EXAMPLE

The inventor presented a method of producing oxidized immunoglobulins through various example embodiments as follows and demonstrated that oxidized immunoglobulin exhibits excellent immunomodulatory effects compared to conventional immunoglobulins as well as excellent immunomodulatory therapeutic effects on diseases associated with immune dysregulation including allergic diseases, chronic inflammatory diseases, autoimmune diseases, and malignant tumor diseases by immune cell culture experiments, animal experiments, and human clinical trials.

### <Example 1> Clinical Trial on an Anticancer Immunotherapeutic Effect of Oxidized Immunoglobulin in Patients with Advanced Solid Tumors

After receiving permission for clinical trials from the Institutional Ethics Committee, the inventor conducted a clinical trial to verify the anticancer immunotherapeutic effect and safety of ‘autologous total IgG intramuscular injection therapy’ in patients with advanced solid tumors (stage 4) with a life expectancy of less than 6 months, and more specifically, including patients with lung cancer, colorectal cancer, ovarian cancer, mesothelioma, breast cancer, gastric cancer, or prostate cancer with cancer metastasis in remote areas and those the disease is not controlled by conventional standard anticancer treatments.

400 ml of venous blood was collected from patients participating in the clinical trial using a double bag for blood donation including anticoagulants before initiation of treatment, and then centrifuged to separate 200 ml of the patient's own plasma. One treatment cycle was defined as that liquid state of autologous IgG (obtained by being purified from the plasma by affinity chromatography using Protein A bead in a sterile and non-toxic condition) was administered by 8 times of intramuscular injections of 50 mg per injection for 4 weeks and having a 4-week resting period after the injections.

At the end of each one treatment cycle, the patient's clinical condition was checked, and the abovely defined one treatment cycle was repeated up to 4 cycles of clinical treatment if the patient's general health condition did not deteriorate to the extent that the clinical trial cannot be continued and the patient agrees to continue the treatment, so as to observe the clinical condition of the solid tumor by examination findings, X-ray examination, computed tomography (CT), magnetic resonance imaging (MRI) examination, radio-isotope examination, and blood tests.

Among the patients, 4 patients who participated in the initial clinical trial were administered intramuscularly with purified autologous total IgG without oxidation, but all 4 patients who participated in the initial clinical trial did not experience a clinically significant therapeutic effect and died within 6 months after the start of the study without completing pre-planned 4 cycles of treatment.

Accordingly, the inventor determined that modification is required for immunoglobulin, which is a material of immunotherapy, to have stronger antigenicity (immunogenicity) in order to obtain a more effective anticancer immunotherapeutic effect than the previous immunoglobulin preparation for malignant tumor patients newly participating in clinical trial.

Accordingly, the inventor designed the present disclosure to enhance the effect of active anti-idiotype immunotherapy by artificially inducing oxidation of immunoglobulin proteins as a method to enhance the immunogenicity of the immunoglobulin proteins and thereby to increase the effect of active anti-idiotype immunotherapy. On the other hand, the industry is currently striving to block oxidation of the immunoglobulin protein as much as possible in the production process of immunoglobulin therapeutic agents (especially monoclonal antibody therapeutic agents), because (1) when oxidized immunoglobulin in therapeutic agent is administered to the human body, oxidized immunoglobulin increase in production of anti-idiotypic antibodies (i.e., anti-drug antibodies) against antigen binding sites of the immunoglobulin and inhibit the action of therapeutic antibodies and (2) oxidation of immunoglobulin increase in the risk of side effects including protein loss and inflammatory reactions due to complement activation by protein aggregation through formation of polymers.

Accordingly, after completion of the initial clinical in 4 patients with advanced solid tumors, a total of 8 patients with advanced solid tumors who newly participated in the clinical trial received intramuscular injection of oxidized autologous IgG 50 mg in liquid state which is produced by mixing 50 mg of autologous IgG in liquid stat with 200 μg of ozone gas obtained from an ozone gas generator connected to high-pressure pure oxygen for medical use) in a syringe (i.e., 4 micrograms of ozone gas per 1 mg of IgG) for a reaction for 5 minutes, and then removing gas. In the 8 patients with solid tumors received with intramuscular injection of oxidized autologous IgG at the same dose, injection interval, and number of injections as the 4 patients with advanced solid tumors who participated in the initial clinical trial.

1-1. Analysis of the Survival Period of Patients with Advanced Solid Tumors

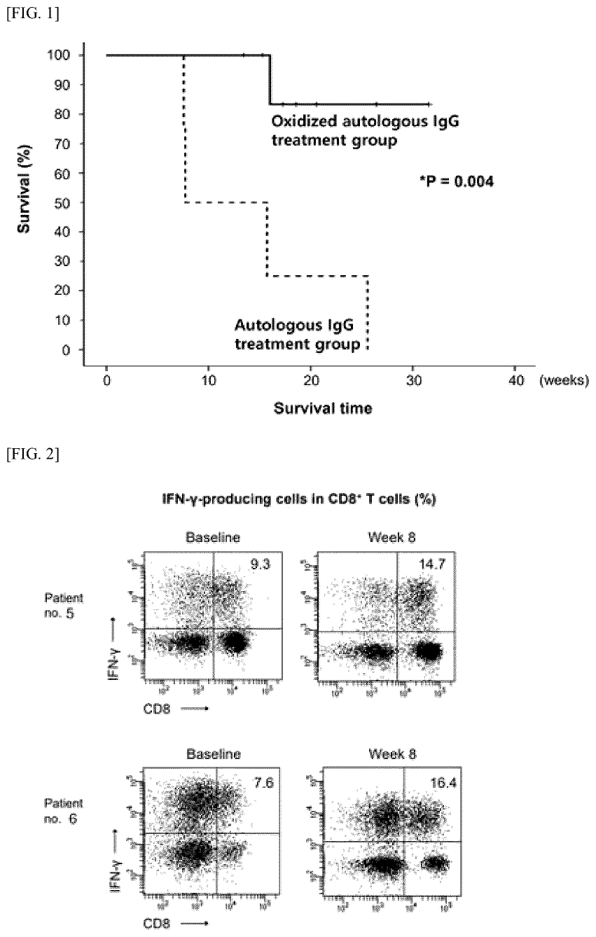

As a result of the clinical trial study, it was found that 8 patients with advanced solid tumors who received intramuscular injection of oxidized autologous IgG (oxidized autologous IgG treatment group) showed a statistically significantly higher survival period, 3-month survival rate, and 6-month survival rate compared to 4 patients with advanced solid cancer (autologous IgG treatment group) who received non-oxidized autologous total IgG (Table 1, FIG. 1). In addition, no severe adverse events related to the clinical study were observed in the course of study from a total of 12 patients with advanced solid tumors who participated in the clinical study.

TABLE 1

| | Number of | | Number of | Number | Number |
|---|---|---|---|---|---|
| | patients | | survivors/ | survivors/ | survivors/ |
| | participating | Median overall | survival | survival | survival |
| | in clinical | survival period, | rate (%) at | rate (%) at | rate (%) at |
| | trials (n) | weeks(95% Ci) | 1 month | 3 months | 6 months |
| Autologous | 4 | 14.2(5.8, 22.5) | 2/50% | 1/25% | 0/0% |
| IgG | | | | | |
| intramuscular | | | | | |
| injection | | | | | |
| group | | | | | |
| Oxidized | 8 | 33.0(28.5, 37.4)* | 0/100% | 0/100%** | 7/86%** |
| autologous | | | | | |
| IgG | | | | | |
| intramuscular | | | | | |
| injection | | | | | |
| group | | | | | |

Kaplan-Meier analysis,
*P < 0.005; Fisher's exact test,
**P < 0.05

Table 1 shows results of analysis of the number of deaths and survival rate between patient group received intramuscular injection of non-oxidized autologous total IG and patient group received intramuscular injection of oxidized autologous total IgG in patients with advanced solid tumors with distant metastasis. The oxidized autologous IgG used in FIG. 1 and Table 1 was produced by reacting IgG with ozone, and as a result of analyzing the number of deaths and survival rate at 1 month, 3 months, and 6 months after the initiation of intramuscular autologous IgG injection, it was found that the survival rate and survival period were statistically significantly increased in patients who received intramuscular injection of oxidized autologous IgG compared to patients who received intramuscular injection of non-oxidized autologous IgG.

1-2. Analysis of Changes in a Proportion (%) of Interferon-Gamma (IFN-γ) Producing Cell Fraction Among Peripheral Blood CD8+ T Cells Before and After Treatment in Patients with Advanced Solid Tumors Who Received Intramuscular Injection of Oxidized Autologous IgG

Flow cytometry analysis was performed by isolating peripheral blood mononuclear cells to observe changes in peripheral blood T cells before and after treatment in 8 patients with advanced solid tumors who received intramuscular injection treatment with oxidized autologous total IgG and participated in the clinical trial study. In a total of 5 patients, except for 3 patients who were unable to collect venous blood for flow cytometry analysis due to deterioration of general condition after participating in the clinical trial, venous blood samples were collected immediately before the start of the 1 treatment cycle (week 0) and at the end of the treatment cycle (week 8), and flow cytometry analysis was performed.

TABLE 2

| | Interferon-gamma (IFN-γ) cells in CD8+ T cells (%) | |
|---|---|---|
| Patients. No | Baseline (week 0) | Week 8 |
| 5 | 9.3 | 14.7 |
| 6 | 7.6 | 16.4 |
| 9 | 5.2 | 4.6 |
| 10 | 50 | 47.7 |
| 11 | 32.4 | 36.3 |

Table 2 shows data on a fraction ratio (%) of cells producing IFN-γ among peripheral blood CD8+ T cells isolated from the blood collected from blood collected immediately before the start of treatment (at week 0), at week, and at 4 weeks after the end of 8 times of oxidized IgG intramuscular injections (at week 8) in 5 patients with advanced solid tumors who received intramuscular injection of oxidized autologous total IgG produced by reacting IgG with ozone. Referring to Table 2, in 3 out of the 5 patients, an increase in the fraction (%) of T cells producing IFN-γ, which is known to play a role in killing cancer cells, was observed among the total peripheral blood CD8+ T cells (cytotoxic T cells). Additionally, among 5 patients who received intramuscular injection of oxidized autologous total IgG, two patients (No. 5 and No. 6 in Table 2) who showed an increase of more than 50% in the fraction ratio (%) of cells producing IFN-γ among peripheral blood CD8+ T cells at the end of the first cycle of treatment (week 8) compared to a baseline (week 0) revealed very good clinical course and systemic condition of patients after the above treatment after completing one cycle of oxidized autologous total IgG intramuscular injection therapy as well as clinically significant antitumor therapeutic effects (2 out of 8 patients, 25% clinical response rate) in which overall tumor size and number of metastatic lesions were maintained without an increase (stable disease state) on imaging tests (CT and MRI) compared to before the start of the treatment, such that a stable disease state in which the tumor did not progress further was maintained continuously throughout the entire observation period, allowing a total of 4 cycles of repeated treatment.

FIG. 2 shows results of flow cytometry analysis of the proportion (%) of cell fraction producing IFN-γ among peripheral blood CD8+ T cells of two patients with advanced solid tumors who received intramuscular injection of oxidized autologous total IgG. The above results were obtained by analysis using blood collected just before the start of oxidized autologous total IgG intramuscular injection (baseline, wee 0) and at the end of the 1 treatment cycle (week 8).

Referring to Table 2 and FIG. 2, it was found that two patients (No. 5, No. 6) who were able to receive the oxidized autologous IgG intramuscular injection treatment with repeated 4 treatment cycles had significant activation of cytotoxic T cells (CD8+ T cells) with the proportion (%) of the cell fraction producing IFN-γ among CD8+ T cells increased by more than 50% at the end of 1 treatment cycle (week 8) of oxidized autologous IgG intramuscular injection compared to the start of treatment (week 0), Thereby, it found that oxidized autologous total IgG intramuscular injection treatment showed an anticancer immunotherapeutic effect through activation of IFN-γ-secreting CD8+ T cells (i.e., cytotoxic T cells) in patients with advanced solid tumors.

Therefore, in the case of pembrolizumab (brand name “Keytruda”, Merk, USA), an anti-PD1 monoclonal antibody therapeutic agent for anti-cancer immunotherapy that has recently been most commonly used in patients with advanced solid tumors worldwide, considering the published results (Chang E, et al. The Oncologist 2021:26:e1786-e1799) that the proportion (clinical response rate) showing significant anti-tumor therapeutic effects, such as maintaining stable disease with no further increase in tumor size or significantly reducing tumor size, is approximately 10%-30% in patients with various types of advanced solid tumors, the fact that significantly longer survival period and high 3-month and 6-month survival rates were shown in the 8 patients with advanced solid cancer who received intramuscular injection of oxidized autologous total IgG observed in Example 1 of the present disclosure, compared to the 4 patients who received intramuscular injection of non-oxidized autologous total IgG as a control group, suggests that ‘the pharmaceutical composition including oxidized immunoglobulin as an active ingredient’ of the present disclosure has clinical utility as an anticancer immunotherapeutic agent in patients with advanced solid cancer. In addition, in the case of existing anti-PD1 antibody therapeutic agents, it is known that autoimmune diseases occur as systemic side effects in a significant number of patients who underwent treatment. However, in the case of intramuscular injection of oxidized immunoglobulins of the present disclosure, no systemic side effects were observed in all treated patients. Therefore, in patients with advanced solid tumors, treatment using the pharmaceutical composition of the present disclosure, compared with anticancer immunotherapy using conventional anti-PD1 antibody, is believed to be a new anti-cancer immunotherapy that has a similar anti-tumor therapeutic effect (clinical response rate) and the advantage of being safer in terms of systemic side effects.

### <Example 2> Method of Preparing a Pharmaceutical Composition Including Oxidized Immunoglobulins as a Main Component-1

Ozone (O₃) and sodium hypochlorite (NaClO), which generate oxygen free radicals, which are most commonly used for oxidizing proteins, were reacted with IgG to induce oxidation of IgG protein, and the oxidation status and degree of oxidation of the IgG were checked by immunoblot. Commercially available immunoglobulins for intramuscular injection (gamma globulin, Green Cross Corporation, Korea) or immunoglobulins for intravenous injection (10% IVglobulin SN, Green Cross Corporation, Korea) purified from the blood of multiple healthy donors were reacted with ozone gas or liquid state of sodium hypochlorite for 5 minutes at room temperature, respectively. After the reaction, to detect oxidized immunoglobulins SDS-PAGE and immunoblot analysis were performed with anti-dinitrophenyl (DNP) antibody using anti-Oxyblot™ Protein Oxidation Detection Kit (Millipore, Billerica, MA, USA), in which kit, using the characteristics of 2,4-dinitrophenylhydrazine (DNPH) that reacts with the carbonyl group produced in the oxidized protein and changes to dinitrophenylhydrazone (DNP-hydrazone) so as.

### 2-1. Analysis of Experimental Materials

Commercially available Immunoglobulin medication for intramuscular injection (gamma globulin, Green Cross Corporation, Korea) prepared from the blood of multiple healthy human donors are described in the manufacturer's drug instruction as including 165 mg/mL of human IgG, and the medication is supplied in a solution form in glass vials for injection.

In order to re-check immunoglobulin components included in the drug, a human immunoglobulin injection agent (gammaglobulin, Green Cross Corporation, Korea) for intramuscular injection was diluted with distilled water at 10 mg/mL, and a concentration of IgG, IgM, IgA, and albumin in the diluted solution was quantified using a nephelometry measuring instrument (Cobas 8000 series, Roche Diagnostics, Mannheim, Germany) and the, measurement showed that 9.41 mg/ml IgG was included while concentrations of IgA, IgM, and albumin were lower than the minimum detection limits of the measurement method using the instrument (IgA<20 mg/dL, IgM<13 mg/dL, albumin <100 mg/dL). In addition, the concentration of IgE measured by fluorescence enzyme immunoassay using the ImmunoCAP assay (Phadia US, Portage, MI, USA) system was measured to be 2.54 KU/L.

### 2-2. Validation of Protein Oxidation Induction Using Commercialized Human Immunoglobulin for Intramuscular Injection

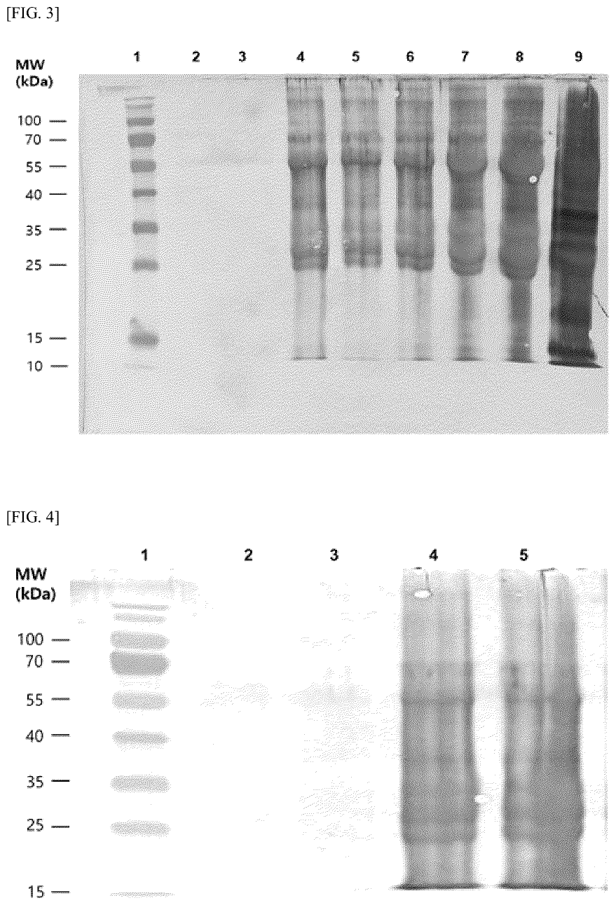

FIG. 3 shows a result of confirming whether the immunoglobulins are oxidized or not by immunoblot analysis using anti-DNP antibody, after carrying out oxidation of commercialized immunoglobulin for intramuscular injection by mixing with ozone or sodium hypochlorite. 1 mg of the immunoglobulin G was oxidized at room temperature for 5 minutes according to the following conditions, and 4 μg of IgG was added for each lane during electrophoresis (SDS-PAGE). The conditions for each lane are: lane 1 is a molecular weight marker, lane 2 is commercialized non-oxidized human immunoglobulin G for intramuscular injection (hIgG), lane 3 is hIgG treated with 0.25 μg of ozone per 1 mg of IgG, lane 4 is hIgG treated with 0.5 μg of ozone per 1 mg of IgG, lane 5 is hIgG treated with 1 μg ozone per 1 mg of IgG, lane 6 is hIgG treated with 2 μg of ozone per 1 mg of IgG, lane 7 is hIgG treated with 4 μg of ozone per 1 mg of IgG, lane 8 is hIgG treated with 8 μg of ozone per 1 mg of IgG, and lane 9 is hIgG treated with 275 μg of liquid sodium hypochlorite for a reaction for 5 minutes per 1 mg of IgG.

FIG. 4 shows a result of verifying, by immunoblot analysis using anti-DNP antibody, whether the immunoglobulins are oxidized or not after carrying out oxidation by mixing commercialized human immunoglobulins for intramuscular injection and human immunoglobulins for intravenous injection with ozone. 1 mg of the immunoglobulin G was mixed with ozone for 5 minutes at room temperature and oxidized according to the following conditions, and 4 μg of IgG was added for each lane during electrophoresis. The conditions for each lane are: lane 1 is a molecular weight marker, lane 2 is non-oxidized commercialized human immunoglobulin G for intramuscular injection, lane 3 is non-oxidized commercialized human immunoglobulin G for intravenous injection, lane 4 is human immunoglobulin G for intramuscular injection oxidized by a reaction with 5 μg of ozone per 1 mg of IgG, and lane 5 is human immunoglobulin G for intravenous injection oxidized by a reaction with 5 μg of ozone per 1 mg of IgG.

Referring to FIGS. 3 and 4, as the experimental results, it was confirmed that significant oxidation of immunoglobulins can be induced by both ozone and sodium hypochlorite, which are known to generate oxygen free radicals. In addition, it was confirmed that the oxidation of immunoglobulins was increased dose-dependently as the amount of treated oxygen free radicals was increased. From the above result, it was found that commercially available immunoglobulin G preparations for intramuscular or intravenous injection included non-oxidized immunoglobulins as a raw material, and when mixed and reacted with ozone or sodium hypochlorite, immunoglobulins can be effectively oxidized.

### <Example 3> Method of Preparing a Pharmaceutical Composition Including Oxidized Immunoglobulins as a Main Component-2

In order to find an appropriate oxidation condition that can minimize the loss of protein due to aggregation and precipitation caused by formation of polymers upon oxidation of immunoglobulins, an amount of protein loss was calculated using <Equation 1> below, by oxidizing immunoglobulins with various degree of oxidation conditions, centrifuging a solution including the oxidized immunoglobulin and then quantifying protein concentration included in the supernatant, and an experiment was conducted to find conditions for immunoglobulin oxidation where appropriate oxidation occurred while minimizing loss due to aggregation of immunoglobulins. Commercially available human IgG for intramuscular injection (gamma globulin, Green Cross Corporation, Korea) was diluted to 10 mg/ml using distilled water, mixed with ozone gas at various concentrations, and reacted at room temperature for 5 minutes. After the reaction, centrifugation was performed at 13,000 g for 15 minutes, and the concentration of immunoglobulins present in the supernatant was measured using Bradford measurement reagent (Biorad, USA). In the present Examples, oxidized IgG in solution state used in the experiments was obtained by mixing IgG in liquid state and ozone gas in a syringe for 5 minutes and then removing the gas from the syringe, wherein the experiment was conducted with four repetitions (quadruplicate) per ozone treatment condition, and the results were expressed as mean±standard deviation to be shown in Table 3 below.

Percentage of loss of IgG protein (%)=(concentration of non-oxidized IgG protein−concentration of IgG protein present in the centrifuged supernatant after oxidation)/non-oxidized concentration of IgG protein×100 <Equation 1>

TABLE 3

| | Concentration of IgG | |
|---|---|---|
| Amount of ozone reacted | present in centrifuged | Loss of IgG |
| with 1 mg of human IgG | supernatant after oxidation | protein due to |
| for 5 minutes | (mg/mL) | oxidation (%) |
| 0 μg | 10.9 ± 0.1 | — |
| 0.5 μg | 10.8 ± 0.03 | 0.9 ± 0.9 |
| 1 μg | 10.4 ± 0.1 | 4.4 ± 1.4 |
| 2 μg | 10.2 ± 0.1 | 5.9 ± 1.9 |
| 4 μg | 10.1 ± 0.2 | 7.6 ± 2.1 |
| 8 μg | 9.0 ± 0.2 | 18.2 ± 0.3 |
| 16 μg | 7.9 ± 0.1 | 27.1 ± 1.1 |

Data are presented as mean±standard deviation from quadruplicate experiments

Table 3 shows results of calculating an loss rate of IgG protein due to aggregation and precipitation of oxidized IgG made by mixing ozone gas with a commercially available human IgG solution for intramuscular injection (purified and prepared from the blood of a large number of healthy human blood donors). Referring to Table 3 above, as shown in the experimental results, the more oxidation is induced (i.e., the more the amount of oxygen radicals reacted), the more the immunoglobulin present in the solution state is aggregated and precipitated. In other words, it may be determined that there is a dose-dependent effect between the degree of oxidation and the aggregation and precipitation of immunoglobulins.

Therefore, it was found that it is reasonable to properly oxidize immunoglobulins to make it a pharmaceutical composition for immunomodulatory therapies under the condition that the oxidation of immunoglobulins may be induced while the loss due to aggregation of immunoglobulin may be minimized. The condition can be that the loss rate of immunoglobulins is less than 10% or the amount of ozone processed per 1 mg of human IgG is 0.1 to 4 μg.

### <Example 4> Checking of Whether Oxidized Immunoglobulins May Exhibit an Immunomodulatory Effect when Administered Intramuscularly in Normal Subjects Via Clinical Trial

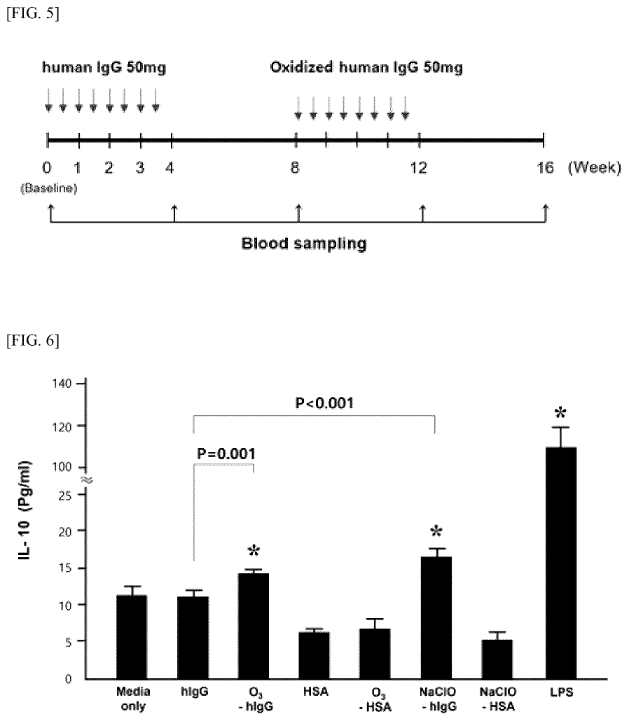

Whether oxidized immunoglobulins can exhibit a better immunotherapeutic effect compared to non-oxidized untreated immunoglobulin proteins even in healthy normal subjects was confirmed by clinical trial. One healthy normal adult human subject received intramuscular injection of 50 mg of commercially available non-oxidized IgG preparation (gamma globulin, Green Cross Corporation, Korea) for intramuscular injection in solution state 8 times for 4 weeks, with 4 weeks of resting period. After having the resting period, 50 mg of a commercially available human IgG preparation for intramuscular injection in liquid form was mixed with 400 μg of ozone in a syringe immediately before intramuscular injection (i.e., 8 μg of ozone per 1 mg of human IgG), a reaction was performed for 5 minutes, and then treatment involving intramuscular injection with oxidized IgG in liquid form (from which ozone gas was removed from the syringe) was performed 8 times for 4 weeks, with another 4 weeks of resting period. The clinical study design for these treatments is shown in FIG. 5. In addition, the serum samples, obtained after collecting the venous blood at 4-week intervals from the time immediately before the first intramuscular injection of human IgG until the 16^{th} week, were stored frozen at −20° C. and thawed again at room temperature, and the concentrations of interleukin-10 (IL-10) and IFN-γ in serum samples were measured using a commercially available enzyme-linked immunosorbent assay (ELISA) measurement kit (BD PharMingen, San Diego, CA, USA), and the results are shown in Table 4.

TABLE 4

| | Concentration of | |
|---|---|---|
| | interleukin-10 in serum | Concentration of interferon- |
| Timepoint | (pg/mL) | gamma in serum (pg/mL) |
| 0 week (Baseline) | 15.5 | Undetectable (<4 pg/mL) |
| 4 weeks | 19.0 | Undetectable (<4 pg/mL) |
| 8 weeks | 18.3 | Undetectable (<4 pg/mL) |
| 12 weeks | 18.9 | Undetectable (<4 pg/mL) |
| 16 weeks | 28.9 | 15.0 |

Table 4 shows results of clinical trials comparing the immunomodulatory effects of intramuscular injections of human IgG and oxidized human IgG in one healthy normal human subject by measuring IL-10 and IFN-γ concentrations in serum samples.

FIG. 5 shows a design of a clinical trial study conducted to determine the difference in the immunomodulatory effect of intramuscular injection of commercially available human IgG and intramuscular injection of oxidized human IgG using ozone in one healthy normal human subject.

Referring to Table 4 and FIG. 5 above, compared with at the timing of week 8 (4 weeks after intramuscular injections of 50 mg of human IgG), the concentrations of IL-10 and IFN-γ in the serum samples were significantly increased at the timing of week 16 (4 weeks after intramuscular injections of 50 mg of oxidized human IgG 8 times for 4 weeks). Therefore, through the results of these clinical trials, it can be confirmed that oxidized immunoglobulins exhibit significantly higher immunomodulatory effects than non-oxidized immunoglobulins even in normal human subject. In addition, no adverse events related to the clinical trial were observed during the clinical trial period in the above normal human subject.

### <Example 5> Validation of the Immunomodulatory Effect of Oxidized Immunoglobulins in Human Peripheral Blood Mononuclear Cells

In order to verify the immunomodulatory effect of oxidized immunoglobulins, a peripheral blood mononuclear cell culture model (that is commonly used to confirm the immunomodulatory efficacy of new drug candidate) was used. Peripheral blood mononuclear cells (PBMCs) separated from the venous blood of normal individuals by density gradient centrifugation were cultured and then reacted by replacement of a culture medium containing immunoglobulins, and then the culture medium was collected to measure concentration of cytokines (which are the main mediators of immune response present in the culture medium) and to compare and analyze the immunomodulatory effects of oxidized immunoglobulins and non-oxidized immunoglobulins.

In 2 healthy normal adults (blood donor 1, blood donor 2), PBMCs isolated from the venous blood of each donor by density gradient centrifugation using Cell Preparation Tubes™ (BD Biosciences, San Jose, CA) were dispensed into 24 well tissue culture plates to meet PBMCs of 5×10⁶ cells in 1 mL of RPMI culture medium including 10% fetal calf serum, 2 mM glutamate, 50 μg/ml penicillin streptomycin, and 10 mM HEPES, followed by culture in CO₂ incubator at 37° C. for 24 hours. After the culture, the culture plate was centrifugated, the existing culture medium was removed, and after culturing again in the CO₂ incubator at 37° C. for 24 hours under the following conditions, the culture plate was centrifuged to collect the culture supernatant for analysis, wherein the conditions are: a case treated only with 1 ml of culture medium per well (negative control; Media only), a case treated with 1 mL of culture medium including 80 μg of human IgG (hIgG), a case treated with 1 mL of culture medium including 80 μg of hIgG reacted with 1 μg of ozone per 1 mg of hIgG for 5 minutes (O₃-hIgG), a case treated with 1 mL of culture medium including 80 μg of human serum albumin (HSA), a case treated with 1 mL of culture medium including 80 μg of HSA reacted with 1 μg of ozone per 1 mg of HSA for 5 minutes (O₃-HSA), a case treated with 1 ml of culture medium including 80 μg of hIgG reacted with 275 μg sodium hypochlorite (NaClO) for 5 minutes per 1 mg of hIgG (NaClO-hIgG), a case treated with 1 mL of culture medium including 80 μg of HSA oxidized by reacting 275 μg per 1 mg of HSA with sodium hypochlorite for 5 minutes (NaClO-HSA), and a case treated with 1 mL of culture medium including 10 ng of lipopolysaccharide (LPS, Sigma-aldrich Co., USA) as a positive control experiment. In the above experiment, the experiment was repeated by 4 times (quadruplicate) for each condition. The supernatant obtained by the above experiment was aliquoted, frozen, and then thawed at room temperature, and the concentration of IL-10 and IL-8 cytokine present in the supernatant was measured using cytokine ELISA set (BD PharMingen, San Diego, CA, USA) according to the manufacturer's recommended method.

### 5-1. Confirmation of the Effect of Increasing IL-10 Concentration in Culture Medium Induced by Oxidized Immunoglobulin in a Peripheral Blood Mononuclear Cell Culture Laboratory Model

TABLE 5

| | | | *Significant | |
|---|---|---|---|---|
| | | | increase | |
| | Stimulants contained | Concentration | compared to | **p-value |
| | in 1 ml of culture | of IL-10 (pg/mL) | media only | compared to |
| Conditions | media/well | (mean ± SEM) | (p < 0.05) | condition 2 |
| 1 | Media only | 11.4 ± 0.6 | | |
| 2 | hIgG 80 μg | 11.2 ± 0.4 | — | |
| 3 | O₃ treated (1 μg O₃/ | 14.4 ± 0.3 | * | 0.001 |
| | 1 mg hIgG) hIgG 80 μg | | | |
| 4 | HSA 80 μg | 6.4 ± 0.2 | — | |
| 5 | O₃ treated (1 μg O₃/ | 7.0 ± 0.6 | — | |
| | 1 mg HSA) HSA 80 μg | | | |
| 6 | NaClO treated (275 | 16.6 ± 0.5 | * | <0.001 |
| | μg NaClO/1 mg | | | |
| | hIgG) hIgG 80 μg | | | |
| 7 | NaClO treated (275 | 5.3 ± 0.4 | — | |
| | μg NaClO/1 mg | | | |
| | HSA) HSA 80 μg | | | |
| 8 | LPS 10 ng | 108.8 ± 4.9 | * | <0.001 |

Interleukin-10 (IL-10); human immunoglobulin G (hIgG); Ozone (O₃); Human serum albumin (HSA); Sodium hypochlorite (NaClO); Lipopolysaccharide (LPS); standard error of the mean (SEM); Data are presented as mean±SEM from quadruplicate experiment. It is considered to have a statistically significant difference if the p value is less than 0.05 in the comparison with *Student t-test (independent sample test). ** p-value is considered to have a statistically significant difference when the p-value is less than 0.05 in the comparison of results of the measurement value in a certain condition with measurement value of the non-oxidized hIgG group (condition 2) by student t-test (independent sample test).

Table 5 shows results of comparing IL-10 concentration in the culture medium collected after reacting human peripheral blood mononuclear cells isolated from peripheral blood of a healthy normal individual (blood donor 1) with human IgG or oxidized human IgG, which is schematized as a graph and shown in FIG. 5.

Referring to Table 5 and FIG. 6, as a result of experiments using the venous blood of healthy normal individuals (blood donor 1), in the case in which immune cells isolated from peripheral blood of healthy normal individuals (blood donor 1) (peripheral blood mononuclear cells; PBMCs) were reacted with human IgG oxidized with ozone or sodium hypochlorite (oxidized hIgG), compared to the case treated only with the culture medium as a negative control (media only) or the case reacted with non-oxidized human IgG, it was found that the IL-10 concentration of the culture supernatant was significantly high (p<0.05). On the other hand, there was no significant increase in the IL-10 concentration of the culture supernatant in the case with the condition in which non-oxidized hIgG or oxidized HSA was treated compared to a case treated with only the culture medium as the negative control.

In addition, when reacted with oxidized human IgG by ozone or sodium hypochlorite (oxidized hIgG), it was confirmed that the IL-10 concentration in the culture supernatant was significantly higher than that reacted with non-oxidized hIgG (p<0.001). In the case treated with LPS used as a positive control, a significantly higher concentration of IL-10 was measured in the culture supernatant compared to other conditions (p<0.05), confirming that this experiment using the cultured human peripheral blood mononuclear cells (PBMCs) was appropriately performed.

TABLE 6

| | | | *Significant | |
|---|---|---|---|---|
| | | | increase | |
| | Stimulants contained | Concentration | compared to | **p-value |
| | in 1 ml of culture | of IL-10(pg/mL) | media only | compared to |
| Conditions | media/well | (mean ± SEM) | (p < 0.05) | condition 2 |
| 1 | Media only | 7.7 ± 0.6 | | |
| 2 | hIgG 80 μg | 7.6 ± 0.7 | — | |
| 3 | O₃ treated (1 μg O₃/ | 17.8 ± 0.5 | * | <0.001 |
| | 1 mg hIgG) hIgG 80 μg | | | |
| 4 | HSA 80 μg | 4.9 ± 0.4 | — | |
| 5 | O₃ treated (1 μg O₃/ | 3.7 ± 0.2 | — | |
| | 1 mg HSA) HSA 80 μg | | | |
| 6 | LPS 10 ng | 455.4 ± 11.2 | * | <0.001 |

Interleukin-10 (IL-10); human immunoglobulin G (hIgG); Ozone (O₃); Human serum albumin (HSA); Sodium hypochlorite (NaClO); Lipopolysaccharide (LPS); standard error of the mean (SEM); Data are presented as mean±SEM from quadruplicate experiment. It is considered to have a statistically significant difference if the p value is less than 0.05 in the comparison with *Student t-test (independent sample test). ** p-value is considered to have a statistically significant difference when the p-value is less than 0.05 in the comparison of results of the measurement value of the non-oxidized hIgG group (condition 2) with measurement value of other conditions by the student t-test (independent sample test).

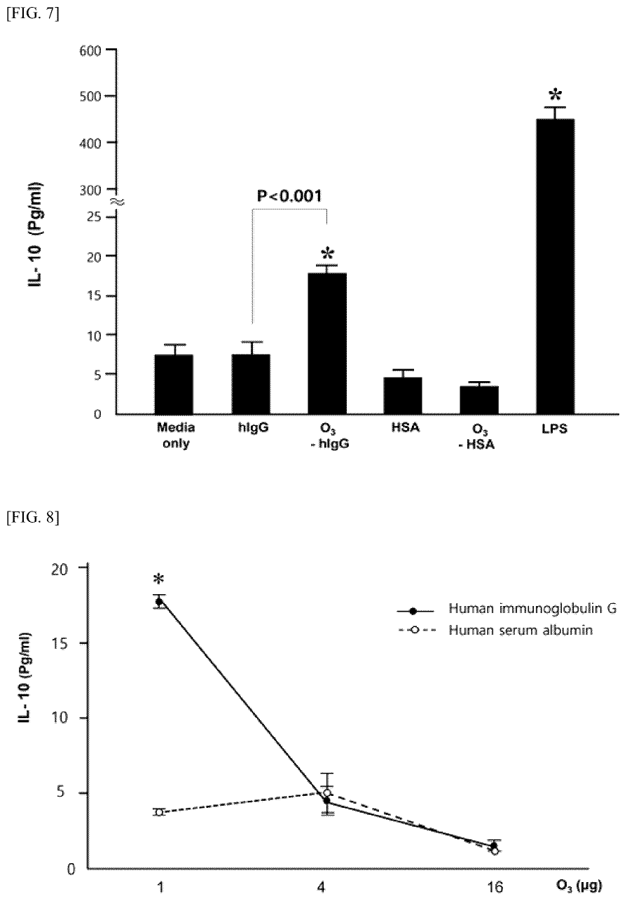

Table 6 shows comparison of the IL-10 concentration in the culture medium collected after reacting IgG or oxidized IgG with human peripheral blood mononuclear cells isolated from peripheral blood of another healthy normal individuals (blood donor 2) and cultured, which is schematized as a graph and shown in FIG. 7.

Referring to Table 6 and FIG. 7, as a result of experiments using peripheral blood of healthy normal individuals (blood donor 2), in the case in which immune cells isolated from healthy normal individuals (peripheral blood mononuclear cells; PBMCs) were reacted with human IgG oxidized with ozone (oxidized hIgG), compared to the case treated only with the culture medium as a negative control (media only) or the case reacted with non-oxidized human IgG, it was confirmed that the IL-10 concentration of the culture supernatant was significantly high (p<0.05). On the other hand, there was no significant increase in the IL-10 concentration of the culture supernatant in the case reacted with non-oxidized hIgG compared to the case treated with only the culture medium as the negative control.

In addition, when reacted with human IgG oxidized with ozone (oxidized hIgG), it was confirmed that the IL-10 concentration in the culture supernatant was significantly higher than that of non-oxidized hIgG (p<0.001). In the case treated with LPS used as a positive control, a significantly higher concentration of IL-10 was measured in the culture supernatant compared to other conditions (p<0.05), suggesting that this experiment using cultured human peripheral blood mononuclear cells (PBMCs) was appropriately performed.

### 5-2. Change in the IL-10 Concentration in a Culture Medium According to Changes in Dose of Ozone to Oxidize Immunoglobulins in Peripheral Blood Mononuclear Cell Culture Laboratory Model

After culturing PBMCs isolated from the venous blood of one healthy normal individual (blood donor 2) in the same manner as above, the culture was performed again in the CO₂ incubator at 37° C. for 24 hours under the following conditions, the culture plate was centrifuged, and the culture supernatant was collected for analysis, wherein the conditions are: a case treated with 1 mL of culture medium including 80 μg of hIgG reacted with 1 μg of ozone for 5 minutes per 1 mg of hIgG per well, a case treated with 1 mL of culture medium including 80 μg of hIgG reacted with 4 μg of ozone for 5 minutes per 1 mg of hIgG, a case treated with 1 mL of culture medium including 80 μg of hIgG reacted with 16 μg of ozone for 5 minutes per 1 mg of hIgG, a case treated with 1 mL of culture medium including 80 μg of HSA reacted with 1 μg of ozone for 5 minutes per 1 mg of HSA, a case treated with 1 mL of culture medium including 80 μg of HSA reacted with 4 μg of ozone for 5 minutes per 1 mg of HSA, and a case treated with 1 mL of culture medium including 80 μg of HSA reacted with 16 μg of ozone for 5 minutes per 1 mg of HSA. In the above experiment, the experiment was carried out by repeated for 4 times (quadruplicate) for each condition. The supernatant obtained by the above experiment was dispensed, frozen at −20° C., and then thawed at room temperature, and the concentration of IL-10 cytokine present in the supernatant was measured using cytokine ELISA set (BD, USA) according to the manufacturer's recommended method.

TABLE 7

| | Concentration of IL-10 (pg/mL) | | |
|---|---|---|---|
| | (mean ± SEM) | | |
| Amount of O₃ | hIgG 80 μg | HSA 80 μg | |
| reacted with 1 mg | treatment | treatment | P |
| of hIgG or HSA | (1 mL/well) | (1 mL/well) | value* |
| 1 μg | 17.8 ± 0.5 | 3.7 ± 0.2 | <0.001* |
| 4 μg | 4.5 ± 0.9 | 5.1 ± 1.3 | 0.754 |
| 16 μg | 1.5 ± 0.4 | 1.1 ± 0.03 | 0.320 |

Interleukin-10 (IL-10); human immunoglobulin G (hIgG); Ozone (O₃); Human serum albumin (HSA); Lipopolysaccharide (LPS); standard error of the mean (SEM); Data are presented as mean±SEM from quadruplicate experiment. It is considered to have a statistically significant difference if the p value is less than 0.05 in the comparison by *Student t-test (independent sample test).

Table 7 shows comparison in the difference in IL-10 concentration in the culture medium according to changes in the degree of oxidation of human IgG by reacting human peripheral blood mononuclear cells isolated from the peripheral blood of a healthy normal individual (blood donor 2) with human IgGs oxidized under various conditions, which was schematized as a graph and shown in FIG. 8.

Referring to Table 7 and FIG. 8, as a result of the experiment, a significant difference in IL-10 concentration in the culture medium was observed between 80 μg of hIgG treated with 1 μg of ozone per 1 mg of human IgG and 80 μg of HSA reacted with 1 μg of ozone per 1 mg of HSA for 5 minutes (*p<0.001, Inter-group differences were analyzed by the Student t-test).

In addition, in the case of oxidized human IgG (hIgG), when compared the case of 80 μg of hIgG treated with 1 μg of ozone per 1 mg of human IgG, to the case of 80 μg of hIgG treated with 4 μg of ozone per 1 mg of hIgG or the case of 80 μg of hIgG treated with 16 μg of ozone per 1 mg of hIgG, it was confirmed that if the dose of ozone for treating hIgG was increased, immunomodulatory effect of oxidized hIgG stimulating secretion of IL-10 was decreased (p<0.05, compared a case treated with 1 μg of ozone per 1 mg of human IgG by Student t-test).

These results indicated that the immunomodulatory effect of the protein may actually be reduced when an amount of ozone reacting with 1 mg of hIgG treating cultured human immune cells was 4 μg or more than 4 μg (i.e., excessive oxidation), under the conditions in which the immunoglobulins were oxidized, as identified in Table 3 of Example 3 in the present disclosure.

### 5-3. Effects of Increasing IL-8 Secretion Induced by Oxidized Immunoglobulins in Peripheral Blood Mononuclear Cell Culture Laboratory Model

TABLE 8

| | | | *Significant | | |
|---|---|---|---|---|---|
| | Stimulants | | increase | | |
| | contained in | Concentration | compared to | p-value | **p-value |
| | 1 ml of culture | of IL-8 (pg/ml) | media only | compared to | compared to |
| Conditions | media/well | (mean ± SEM) | (p < 0.05) | media only | condition 2 |
| 1 | Media only | 107.6 ± 0.8 | | | |
| 2 | hIgG 80 μg | 83.9 ± 2.0 | — | <0.001 | |
| 3 | O₃ treated (1 | 132.5 ± 0.7 | * | <0.001 | <0.001 |
| | μg O₃/1 mg | | | | |
| | hIgG) hIgG | | | | |
| | 80 μg | | | | |
| 4 | HSA 80 μg | 94.3 ± 2.0 | — | 0.001 | |
| 5 | NaClO treated | 252.7 ± 2.8 | * | <0.001 | <0.001 |
| | (275 μg | | | | |
| | NaClO/1 mg | | | | |
| | hIgG) hIgG | | | | |
| | 80 μg | | | | |
| 6 | LPS 10 ng | 253.9 ± 2.8 | * | <0.001 | <0.001 |

Interleukin-10 (IL-10); human immunoglobulin G (hIgG); Ozone (O₃); Human serum albumin (HSA); Sodium hypochlorite (NaClO); Lipopolysaccharide (LPS); standard error of the mean (SEM); Data are presented as mean±SEM from quadruplicate experiment. It is considered to have a statistically significant difference if the p value is less than 0.05 in the comparison with *Student t-test (independent sample test). ** p-value is considered to have a statistically significant difference when the p-value is less than 0.05 in the comparison of results of the measurement value of the non-oxidized hIgG group (condition 2) by the student t-test (independent sample test).

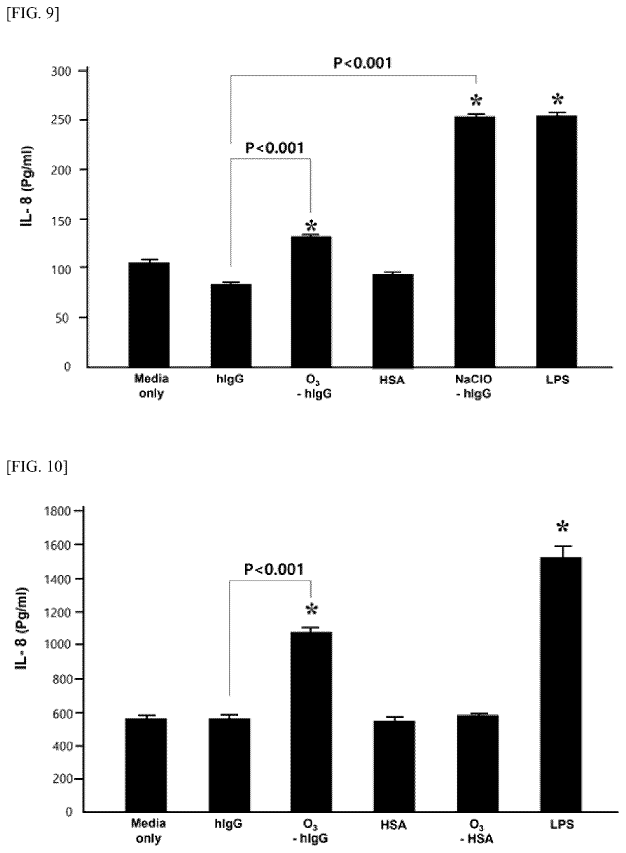

Table 8 shows comparison of IL-8 concentration in the culture medium collected after reacting human peripheral blood mononuclear cells cultured after being isolated from peripheral blood of healthy a normal human subject (blood donor 1) with human IgG or oxidized human IgG, which is schematized as a graph and shown in FIG. 9.

Referring to Table 8 and FIG. 9, as a result of experiments using the peripheral blood of a healthy normal human subject (blood donor 1), immune cells (peripheral blood mononuclear cells; PBMCs) isolated from the peripheral blood of a healthy normal human subject (blood donor 1) under conditions of a case treated with human IgG oxidized with ozone or sodium hypochlorite (oxidized hIgG) produced significantly increased concentrations of IL-8 in the culture medium compared to the case treated only with the culture medium as the negative control or the case reacted with non-oxidized human IgG (p<0.05). On the other hand, it was found that the condition with treatment with non-oxidized hIgG and HSA had no significant increase in the IL-8 concentration in the cell culture supernatant compared to a case treated only with the culture medium as the negative control.

In addition, when LPS used as the positive control was treated, it was confirmed that a significantly higher concentration of IL-8 was secreted in the cell culture supernatant compared to other conditions (p<0.05), and this confirmed that the experiment using the cultured human peripheral blood mononuclear cells (PBMCs) was appropriately performed.

TABLE 9

| | | | *Significant | |
|---|---|---|---|---|
| | Stimulants contained | Concentration of IL-8 | increase compared | **p-value |
| | in 1 ml of culture | (mean ± SEM) | to media only | compared to |
| Conditions | media/well | (pg/mL) | (p < 0.05) | condition 2 |
| 1 | Media only | 556.6 ± 24.2 | | |
| 2 | hIgG 80 μg | 563.7 ± 20.4 | — | |
| 3 | O₃ treated (1 μg O₃/1 | 1079.7 ± 26.3 | * | <0.001 |
| | mg hIgG) hIgG 80 μg | | | |
| 4 | HSA 80 μg | 552.0 ± 19.6 | — | |
| 5 | O₃ treated (1 μg O₃/1 | 581.2 ± 8.5 | — | |
| | mg HSA) HSA 80 μg | | | |
| 6 | LPS 10 ng | 1528.3 ± 68.8 | * | <0.001 |

Interleukin-8 (IL-8); human immunoglobulin G (hIgG); Ozone (O₃); Human serum albumin (HSA); Lipopolysaccharide (LPS); standard error of the mean (SEM); Data are presented as mean±SEM from quadruplicate experiment. It is considered to have a statistically significant difference if the p value is less than 0.05 in the comparison by *Student t-test (independent sample test). ** p-value is considered to have a statistically significant difference when the p-value is less than 0.05 in the comparison of results of the measurement value of the non-oxidized hIgG group (condition 2) by the student t-test (independent sample test).

Table 9 is a comparison of IL-8 concentration in the culture medium collected after reacting cultured human peripheral blood mononuclear cells isolated from the peripheral blood of another healthy normal human subject (blood donor 2) with human IgG or oxidized human IgG, which is schematized as a graph and shown in FIG. 10.

In 5 Referring to Table 9 and FIG. 10, as a result of experiments using the peripheral blood of healthy normal human subject (blood donor 2), immune cells (peripheral blood mononuclear cells; PBMCs) isolated from the peripheral blood of healthy normal individuals (blood donor 2) produced significantly increased concentrations of IL-8 in the culture medium when treated with human IgG oxidized with ozone (oxidized hIgG) compared to the case treated only with the culture medium as the negative control or the case treated with non-10 oxidized human IgG (p<0.05).

In addition, it was confirmed that a significantly higher concentration of IL-8 was secreted (p<0.05) in the culture medium treated with LPS used as the positive control compared to other treatment conditions, and thereby confirming that the experiment using the cultured human peripheral blood mononuclear cells (PBMCs) was appropriately performed.

5-4. Changes in an Amount of IL-8 Secretion in the Culture Medium According to the Change in the Capacity of Ozone that Oxidizes Immunoglobulins in a Peripheral Blood Mononuclear Cell Culture Laboratory Model

After culturing PBMCs isolated from the venous blood of a healthy normal human subject (blood donor 2) in the same manner as above, culture was performed again in the CO₂ incubator at 37° C. for 24 hours under the following conditions, the culture plate was centrifuged, and the culture supernatant was collected for analysis, wherein the conditions are: a case treated with 1 mL of culture medium including 80 μg of hIgG reacted with 1 μg of ozone per 1 mg of hIgG for 5 minutes per well, a case treated with 1 mL of culture medium including 80 μg of hIgG reacted with 4 μg of ozone per 1 mg of hIgG for 5 minutes, a case treated with 1 mL of culture medium including 80 μg of hIgG reacted with 16 μg of ozone per 1 mg of hIgG for 5 minutes, a case treated with 1 mL of culture medium including 80 μg of HSA reacted with 1 μg of ozone per 1 mg of HSA for 5 minutes, a case treated with 1 mL of culture medium including 80 μg of HSA reacted with 4 μg of ozone per 1 mg of HSA for 5 minutes, and a case treated with 1 mL of culture medium including 80 μg of HSA reacted with 16 μg of ozone per 1 mg of HSA for 5 minutes, In the above experiment, the experiment was performed by repeated for 4 times (quadruplicate) per condition. The supernatant obtained by the above experiment was dispensed, frozen at −20° C., and thawed at room temperature, and the concentration of IL-8 cytokine present in the supernatant was measured using cytokine ELISA set (BD, USA) according to the manufacturer's recommended method.

TABLE 10

| | Concentration of IL-8(pg/mL) | | |
|---|---|---|---|
| | (mean ± SEM) | | |
| Amount of O₃ | hIgG 80 μg | HSA 80 μg | |
| reacted with 1 mg | treatment | treatment | P |
| of hIgG or HSA | (1 mL/well) | (1 mL/well) | value* |
| 1 μg | 1079.9 ± 26.3 | 581.2 ± 8.5 | <0.001 |
| 4 μg | 975.3 ± 20.8 | 583.6 ± 12.4 | <0.001 |
| 16 μg | 943.8 ± 49.2 | 597.4 ± 10.3 | <0.001 |

Interleukin-8 (IL-8); human immunoglobulin G (hIgG); Ozone (O₃); Human serum albumin (HSA); Lipopolysaccharide (LPS); standard error of the mean (SEM); Data are presented as mean±SEM from quadruplicate experiment. * It is considered to have a statistically significant difference for the concentration of IL-8 present in the culture supernatant between the hIgG treated group and the HSA treated group treated with different ozone doses when the p value in the comparison by student t-test (independent sample test) is less than 0.05.

Table 10 shows comparison of IL-8 concentration in the culture medium according to the change in the degree of oxidation of human IgG by reacting human peripheral blood mononuclear cells isolated from the peripheral blood of a healthy normal human subject (blood donor 2) and human IgGs oxidized under various conditions, which is schematized and shown in FIG. 11.

Referring to Table 10 and FIG. 11, as results of comparing the same three conditions (1 μg, 4 μg, and 16 μg treated to 1 mg of IgG or 1 mg of HSA, respectively) treated with different doses of ozone for IgG or HSA, the cases treated with oxidized IgG showed significantly high concentrations of IL-8 in the culture medium in all three conditions compared to the cases treated with oxidized HSA (*p<0.001, Inter-group differences were analyzed by the Student t-test).

In addition, in the case of oxidized IgG, it was found that the case of 80 μg of IgG treated with 1 μg of ozone of per 1 mg of IgG had significantly increased immunomodulatory effects to stimulate secretion of IL-8 compared to the case of 80 μg of IgG treated with 4 μg of ozone of per 1 mg of IgG (p<0.05).

These results indicated that when the amount of ozone reacting with 1 mg of hIgG (under the condition of oxidizing the immunoglobulin of the present disclosure) is 4 μg or more, an excessive amount of ozone treating the immunoglobulin can decrease the immunomodulatory effects.

Through this Example 5, it was shown that the oxidized immunoglobulin exhibited a significant immunomodulatory effect. Specifically, it was shown that oxidized immunoglobulins exhibited the immunomodulatory effect inducing secretion of significantly higher amount of IL-10 from human peripheral blood immune cells compared with non-oxidized immunoglobulins, human serum albumin, or oxidized human serum albumin.

The IL-10 is an important immunomodulatory substance that has already been confirmed in a number of studies to exhibit immunomodulatory, anti-inflammatory, anti-allergic, and anticancer effects and it can improve diseases associated with immune dysregulation including autoimmune diseases, cancer, chronic inflammatory diseases, and autoimmune diseases (Saralva M, et al. J Exp Med. 2020; 217:e20190418, Akdis C A, et al. J Clin Invest 2014:124:4678-80). In particular, clinical studies have reported that administration of recombinant IL-10 has induced clinically significant improvements in patients suffering from chronic inflammatory diseases including chronic inflammatory bowel disease, rheumatoid arthritis, and psoriasis as well as advanced solid cancer, and it is under development as new therapeutic agents for the above diseases. On the other hand, in patients with advanced solid cancer, daily administration of pegylated recombinant human IL-10, in which PEGylation is a technology that increases the half-life of protein drugs in the body, has shown a systemic immune enhancing effect with a significant increase in the concentration of IFN-γ in serum specimens collected before and after treatment and a significant increase in the number of CD8+ T cells infiltrating cancer tissues in addition to the prolongation of progression-free survival (Naing A, et al. J Clin Oncol 2016, Naing A, et al. Cancer Cell 2018:34:775-79). Therefore, the Example 5 may demonstrate that oxidized human immunoglobulins showed an immunomodulatory therapeutic effect, an anticancer immunotherapeutic effect, and an anti-allergic immunomodulatory effect increasing secretion of IL-10 from immune cells that can improve patients suffering from chronic inflammatory diseases, malignant tumors, and allergic diseases.

In addition, through the immunomodulatory effect to increase secretion of IL-10 from immune cells by the oxidized immunoglobulin confirmed in Example 5 of the present disclosure, intramuscular injection of the oxidized immunoglobulin (and as also determined through clinical trials in patients with advanced solid tumors in Example 1 of the present disclosure) can induce a systemic immunomodulatory effect increasing the fraction of cells secreting IFN-γ among peripheral blood CD8+ T cells (cytotoxic T cells), thereby produce anticancer immunotherapeutic effects.

In addition, the oxidized immunoglobulin (as shown in Example 5) produced the immunomodulatory effect secreting significantly higher levels of IL-8 from human peripheral blood immune cells, compared to non-oxidized immunoglobulin, human serum albumin, or oxidized human serum albumin. It has been reported that IL-8 exhibits an anti-allergic immunomodulatory effect to inhibit histamine release from basophils, which play an important role in the pathogenesis of allergic diseases (Kuna P et al. J Immunol 1991:147:1920-1924, Alam R, et al. Am J Respir Cell MolBiol 1992:7:427-433). Therefore, through the Example 5, it is evident that the oxidized immunoglobulin showed an antiallergic immunomodulatory effect that can treat allergic diseases.

### <Example 6> Confirmation of an Antiallergic Immunomodulatory Effect of Oxidized Immunoglobulins in an Ovalbumin-Allergy Mouse Model

The anti-allergic immunomodulatory effect of the oxidized immunoglobulin of the present disclosure was verified using an ovalbumin (OVA) allergy mouse model which is currently the most commonly used representative animal model to verify an anti-allergic effect of drugs when developing new drugs for allergic diseases in the industry (Ayoub M, et al. Int Immunopharmacol 2003:3:523-53).

In order to make an OVA allergy mouse model, BALB/c female mice were intraperitoneally injected with a mixture obtained by mixing 0.2 mg/mL OVA (Sigma Chemical Co., St. Louis, Mo) and 20 mg/mL aluminium hydroxide (Thermo scientific, Imject Alum, 77161) at 1:1 (v/v) ratior, and then 0.2 mL of the mixture was administered by three times of intraperitoneal injections on days 0, 7, and 14 to produce OVA allergy. Development of antibodies against OVA was determined by measuring serum OVA-specific IgG antibodies with enzyme-linked immunosorbent assay (ELISA) using serum samples obtained by collecting blood from the heart with a scarification of mice at day 21 and then centrifuging the blood. It was intended to determine whether the oxidized immunoglobulin protein in the allergic animal model could produce a significantly higher anti-allergic immunomodulatory effect compared to non-oxidized human immunoglobulins used as the control.

Specifically, in order to proceed with animal experiments, the OVA-allergy mouse model was divided into a total of 3 groups, and using 18 animals with 6 animals per group, the following treatments were performed three times over days 0, 7, and 14. At this time, Group 1 received subcutaneous injection of 0.2 mL of saline as a negative control group, Group 2 received subcutaneous injection of 10 mg of human immunoglobulin (gamma globulin, Green Cross Corporation, Korea) (hIgG) for intramuscular injection, and Group 3 received subcutaneous injection of 10 mg of liquid oxidized human immunoglobulin (O₃-hIgG) obtained by mixing 4 μg of ozone gas per 1 mg of liquid state of immunoglobulins in a syringe for a reaction for 5 minutes and then removing gas. In order to compare the immunomodulatory effect of oxidized human immunoglobulin subcutaneous injection and non-oxidized human immunoglobulin subcutaneous injection in an OVA allergy mouse model, serum samples were collected from the blood samples collected on the 21^{st} day of mice in each group by centrifugation, and then aliquoted and frozen at −20° C. The titers of IgG specifically reacting with OVA in serum specimens thawed at room temperature were measured by enzyme linked immune-sorbent assay (ELISA). 0.25 μg OVA per well was diluted in a carbonate buffer in a 96 well ELISA plate and subjected to a reaction at 4° C. for 16 hours, a reaction was performed with phosphate buffered saline (PBS) including 3% BSA-0.1% Tween-20 at room temperature for 2 hours to block nonspecific reactions, and mouse serum diluted to 1:500 (v:v) with the same buffer was dispensed by quadruplicate at 100 μl per well, followed by a reaction overnight at 4° C. After reacting the anti-mouse IgG antibody attached with alkaline phosphatase again for 2 hours, the absorbance was measured at 405 nm by color development with p-nitrophenyl phosphate. Between each reaction step, washing was performed 5 times with PBS including 0.1% Tween-20 (PBST). Additionally, the titers of OVA-specific IgG antibodies were expressed by absorbance values measured at 405 nm.

TABLE 11

| | | Titer of OVA- | *p-value compared | |
|---|---|---|---|---|
| | | specific IgG | to condition 1 | **ANOVA p- |
| | | (Absorbance | (Saline treated | value(statistical |
| | Treatments(subcutaneous | value) | OVA-sensitized | differences among |
| Groups | injection) | (mean ± SEM) | mice) | 3 groups) |
| 1 | Saline | 0.295 ± 0.025 | | 0.001 (Tukey's |
| 2 | hIgG 10 mg | 0.252 ± 0.016 | 0.186 | post hoc analysis |
| 3 | O₃ treated (4 μg O₃ / | 0.395 ± 0.022 | 0.013 | Group 1-Group 3: |
| | 1 mg hIgG) hIgG 10 mg | | | 0.013 |
| | | | | Group 2-Group 3: |
| | | | | 0.001) |

human immunoglobulin G (hIgG); ovalbumin (OVA); Ozone (O₃); standard error of the mean (SEM); Data are presented as mean±SEM from quadruplicate experiment. *For p-value, it is considered to have a statistically significant difference when the p value is less than 0.05 in the comparison with results of OVA-specific IgG antibody measurement in the negative control mouse group injected subcutaneously with normal saline by student t-test (independent sample test). ** For p-value, if the p value is less than 0.05 in comparison with the one-way ANOVA test, there is a statistically significant difference in the mean value of OVA-specific IgG antibody among the three groups.

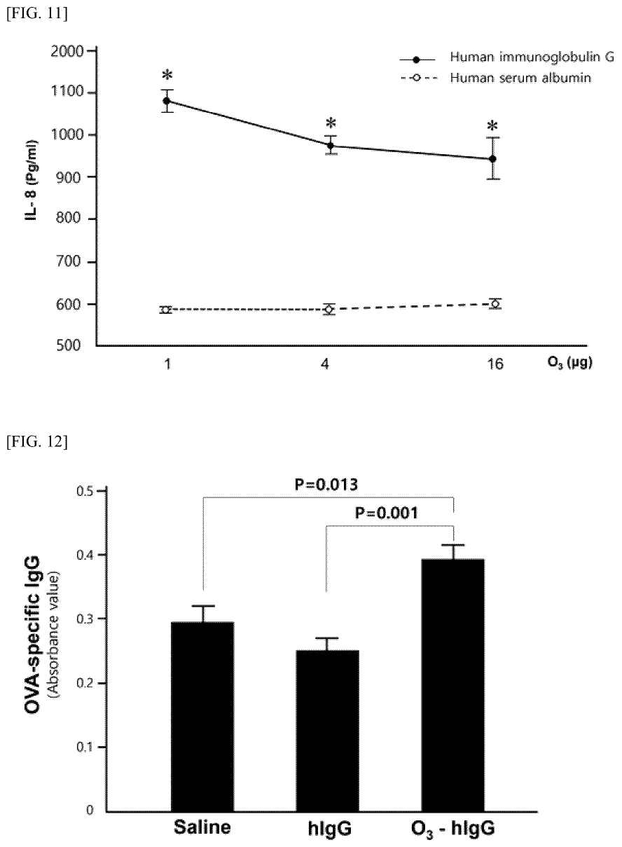

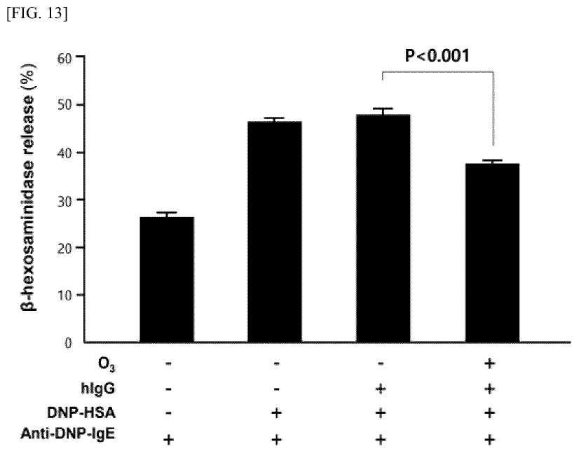

Table 11 shows results of verifying the OVA-specific IgG antibody inducing effect (i.e., antiallergic effect) of hIgG in an ovalbumin (OVA) allergy mouse model, and the same is shown in FIG. 12.

Referring to Table 11 and FIG. 12, as a result of the experiment, a significant difference in the mean value of OVA-specific IgG antibody values was observed between the three groups. As a result of Tukey's post-hoc analysis to determine a significant difference between which groups in the mean value of OVA-specific IgG antibody titers, it was found that in the mouse group administered with 10 mg of oxidized hIgG had significantly higher average value of OVA-specific IgG antibody titers compared to the group of mice administered only with normal saline as the negative control or the group administered with 10 mg of non-oxidized hIgG (One-way ANOVA test with Tukey's post hoc test, p=0.013, p=0.001, respectively). In particular, the group administered with 10 mg of non-oxidized hIgG did not have a statistically significant difference compared to the negative control mouse group administered with normal saline only (p=0.186, Table 11, FIG. 12). Thus, through the Example 6, it is evident that an anti-allergic immunomodulatory effect increasing OVA-specific IgG antibody titer developed when administering oxidized IgG compared to non-oxidized IgG.

Allergen-specific IgG antibodies have been known to act as blocking antibodies that competitively block bindings of allergen-specific IgE antibodies to allergens. In past animal model studies and recent clinical trials in patients with allergy, it was clearly proven and reported that administration of allergen-specific IgG by injection produced a significant anti-allergic effect to suppress allergic reactions caused by binding of allergens and IgE and thereby could provide a therapeutic effect of reducing symptoms of allergic diseases (Flicker S, et al. Curr Top Microbiol Immunol 2011; Gevaert P, et al. J Allergy Clin Immunol 2022). Thus, as shown in Example 6, the allergen-specific IgG production-inducing effect of oxidized hIgG can be applied to treatment of allergic diseases by inducing a meaningful anti-allergic immunomodulatory effect of oxidized IgG.

### Example 7

### Preparation Example 7-1

12 mg of oxidized immunoglobulin (or IgG)

Including one or more immunoadjuvants among immunoadjuvants (0.001-2 mg of aluminum hydroxide, appropriate amount of calcium phosphate, 1 mg of tyrosine, 1 mg of MPL)
4 mg of sodium chloride
45 mg of aminoacetic acid
4 mg of D-mannitol
Appropriate amount of sodium hydroxide
0.8-2 ml of injection water
(Injection water is supplied in a separate vial different from the other ingredients.)

### Preparation Example 7-2

12 mg of oxidized immunoglobulin (or IgG)
0.15 μg of histamine dihydrochloride
4 mg of sodium chloride
45 mg of aminoacetic acid
4 mg of D-mannitol
Appropriate amount of sodium hydroxide
0.8-2 ml of injection water
(Injection water is supplied in a separate vial different from the other ingredients.)

In order to enhance the immunomodulatory effect of oxidized immunoglobulins as described in the above Preparation Examples 7-1 or 7-2, a pharmaceutical composition may be prepared by further mixing an immunoadjuvant or histamine as is commonly practiced in the art.

### <Example 8> Confirmation of an Inhibitory Effect of Oxidized IgG on Immediate-Type Allergic Reaction Caused by Binding of IgE and Antigen in Cultured Rat Basophilic Leukemia Cell Lines

RBL-2H3 cell lines (rat basophilic leukemia cell lines) are commonly used to verify the anti-allergic effects of new drug candidates. The RBL-2H3 cell line is a cell known to have a receptor (FcεRI) of an IgE antibody on the surface, and when activated with IgE and antigen, degranulation from RBL-2H2 cell is induced to secrete a mediator of an allergic reaction, and β-hexosaminidase is known as an indicator substance of degranulation (Planta Med. 1998:64:577-578).

In order to determine whether oxidized IgG can inhibit a development of immediate-type allergic reaction caused by binding of IgE and antigen in basophils, the release of β-hexosaminidase by antigen stimulation after IgE sensitization in RBL-2H3 cell line was measured by the following experimental method.

RBL-2H3 cells were cultured at 37° C. in a moist CO₂ incubator (5% CO₂/95% air) by adding 0.5 mL of cell culture medium supplemented with 10% fetal bovine serum (FBS), 100 U/mL penicillin, and 100 μg/mL streptomycin to Dulbecco's modified eagle medium per well. RBL-2H3 cells were dispensed into a 48-well cell culture plate with 2×10⁵ cells/well, cultured for 24 hours, replaced with 0.5 mL of a medium including 20 ng/ml of dinitrophenyl (DNP)-specific IgE antibody (DNP-sIgE; Sigma, St. Louis, MO, USA) per well, and cultured for 16 hours to sensitize DNP-sIgE on the cells. After cells in each well were washed two times with Siraganian buffer (119 mM NaCl, 5 mM KCl, 1 mM CaCl₂, 0.4 mM MgCl₂, 25 mM PIPES, 5.6 mM glucose, 0.1% bovine serum albumin, pH 7.2p; hereinafter abbreviated as S buffer), treated was 20 μl of S buffer including 2 mg of human IgG as an inhibitor (hIgG; commercially available human IgG preparation for intramuscular injection in a liquid form, gamma globulin, Green Cross Corporation, Korea) or 2 mg of oxidized human IgG (2 mg of O₃-treated IgG; oxidized human IgG obtained by mixing 1 μg of ozone gas per 1 mg of commercially available liquid human IgG for intramuscular injection in a syringe, reacting for 5 minutes, then removing gas for oxidation), wherein under the negative control and positive control experimental conditions, only 20 μl of S buffer was treated and then all four conditions were subjected to reactions at 37° C. for 50 minutes. Afterwards, 180 μl of S buffer was added in the negative control experimental conditions while 180 μl of S buffer including 20 ng of DNP-human serum albumin (DNP-HSA, Sigma, St. Louis, MO, USA) per well was added for the remaining three experimental conditions (As a result, each well ultimately includes 200 μl of buffer), stimulation was performed for 40 minutes, and then the reaction was stopped by cooling the 48-well cell culture plate on ice for 10 minutes. For all four treatment conditions, the results were measured by performing four quadruplicated experiments per condition.

In order to measure the amount of β-hexosaminidase released in the supernatant in each well after the antigen stimulation was completed as above, 50 μL of supernatant was transferred to well of a 96-well plate, 50 μL of substrate buffer (4-p-nitrophenyl-N-acetyl-β-D-glucosaminide, 3.3 mM, sodium citrate, 0.1 M, pH 4.5) was added to the well, a reaction was carried out at 37° C. for 70 minutes, 100 μL of a stop solution (0.1 M Na₂CO₃, pH 10) was added to the well for stopping the reaction, and the absorbance was measured at 405 nm. In addition, in order to measure the amount of β-hexosaminidase remaining in the cell pellet, after removing all the supernatant from each well after the antigen stimulation was completed, 200 μl of 1% Triton X-100 buffer per well was treated in the cell pellet, 50 μL of cell lysate obtained by mixing with a pipette was transferred to well of a 96-well plate, 50 μL of substrate buffer (4-p-nitrophenyl-N-acetyl-β-D-glucosaminide, 3.3 mM, sodium citrate, 0.1 M, pH 4.5) was added to the well to react at 37° C. for 70 minutes, 100 μL of stop solution (0.1 M Na₂CO₃, pH 10) was added to the well for stopping the reaction, and the absorbance was measured at 405 nm.

The degree of β-hexosaminidase release (%) from RBL-2H3 cells was calculated according to the following <Equation 2> as commonly used in previously published papers (Uermosi C, et al. Allergy. 2014; 69:338-347).

β-hexosaminidase % release=[absorbance of supernatant/(absorbance of supernatant+absorbance of cell pellet)]×100 <Equation 2>

In this Example, after sensitization with DNP-specific IgE, to verify the effect of human IgG or oxidized human IgG on the secretion of β-hexosaminidase (an indicator of mast cell degranulation in RBL-2H3 cells stimulated with DNP-HSA antigen), the release rate (% release) of β-hexosaminidase calculated by the above calculation formula was analyzed and compared.

TABLE 12

| | | β-hexosaminidase | *ANOVA p-value |
|---|---|---|---|
| | | release (%) | (statistical differences |
| Conditions | Stimulants contained | (mean ± SEM) | among 4 conditions) |
| 1 | Negative control(DNP- | 26.44 ± 0.84 | <0.001(Tukey's post hoc |
| | sIgE+, DNP-HSA−) | | analysis showed a |
| 2 | Positive control(DNP- | 46.48 ± 0.72 | significant difference |
| | sIgE+, DNP-HSA+) | | between condition 3 and |
| 3 | hIgG 2 mg(DNP-sIgE+, | 47.81 ± 1.34 | condition 4: p < 0.001) |
| | DNP-HSA+) | | |
| 4 | O₃ treated hIgG 2 | 37.81 ± 0.39 | |
| | mg(DNP-sIgE+, DNP- | | |
| | HSA+) | | |

human immunoglobulin G (hIgG); Ozone (O₃); dinitrophenyl-specific IgE (DNP-sIgE); dinitrophenyl-human serum albumin (DNP-HSA); standard error of the mean (SEM); Data are presented as mean=SEM from quadruplicate experiment. * For p-value, it is considered to have a statistically significant difference in the mean value of β-hexosaminidase release rate (% release) among four groups if the p value is less than 0.05 in the comparison with a one-way ANOVA test.

Table 12 shows a result of comparative analysis of a difference in β-hexosaminidase release rate (% release) by IgE-antigen-mediated stimulation when human IgG and oxidized human IgG were treated in rat basophilic leukemia cells, which is schematized and shown in FIG. 13.

Referring to Table 12 and FIG. 13 above, a statistically significant difference in the mean values of the β-hexosaminidase release rate (% release) was observed in the above four experimental conditions (p<0.001). As a result of conducting the Tukey's post-hoc analysis, it was found that the mean value of release rate of β-hexosaminidase was significantly low in the case treated with oxidized human IgG compared with the case treated with human IgG (One-way ANOVA test with Tukey's post-hoc test, p<0.001). In other words, it was confirmed that oxidized human IgG effectively inhibited degranulation from basophilic cells caused by IgE-antigen binding. The experimental results in this Example confirmed that oxidized IgG exhibited an antiallergic effect to suppress IgE-mediated immediate type allergic reaction more effectively than non-oxidized human IgG. In addition, the above experimental results are proving that the oxidized immunoglobulin of the present disclosure is a substance having an anti-allergic effect useful for preventing or treating allergic diseases.

When the pharmaceutical composition including the oxidized immunoglobulin of the present disclosure as an active ingredient is administered to mammals, it is possible to prevent or treat diseases associated immune dysregulation compared with conventional immunoglobulin preparations more effectively and produce new pharmaceutical compositions for preventing and treating diseases associated immune dysregulation.

Having described in detail a specific part of the contents of the present disclosure above, it will be apparent that, for a person skilled in the art, such a specific technique is only a preferred embodiment, and the scope of the present disclosure is not limited thereby. Thus, the substantive scope of the present disclosure will be defined by the attached claims and their equivalents.

1. A method of preventing or treating diseases associated with immune dysregulation in a subject, comprising:
administering a pharmaceutical composition comprising oxidized immunoglobulin as an active ingredient to the subject.

2. The method of claim 1, wherein the oxidized immunoglobulin is obtained by mixing and reacting immunoglobulin with one or more oxidants from the group consisting of oxidants including ozone (O₃), hydrogen peroxide (H₂O₂), and sodium hypochlorite (NaClO).

3. The method of claim 2, wherein the oxidized immunoglobulin is obtained by mixing and reacting 0.1 to 4 μg of ozone per 1 mg of immunoglobulin.

4. The method of claim 1, wherein the immunoglobulin is any one from the group consisting of IgG, IgA, IgM, IgD, and IgE.

5. The method of claim 4, wherein the immunoglobulin is IgG.

6. The method of claim 5, wherein the IgG is an immunoglobulin G isolated from blood of a mammal.

7. The method of claim 6, wherein the IgG is IgG isolated from the blood of the mammal itself or IgG isolated from the blood of another mammal.

8. The method of claim 1, wherein the disease associated with immune dysregulation is any one from the group consisting of allergic diseases, chronic inflammatory diseases, autoimmune diseases, and malignant tumor diseases.

9. The method of claim 8, wherein the allergic disease is any one from the group consisting of bronchial asthma, allergic rhinitis, allergic conjunctivitis, atopic dermatitis, urticaria, food allergy, anaphylaxis, and drug allergy.

10. The method of claim 8, wherein the chronic inflammatory disease or autoimmune disease is any one from the group consisting of degenerative arthritis, chronic inflammatory gastroenteritis, ankylosing spondylitis, chronic pustular dermatitis, rheumatoid arthritis, systemic lupus erythematosus, pemphigus, autoimmune thyroiditis, autoimmune hepatitis, chronic inflammatory bowel disease, autoimmune nephritis, chronic inflammatory gastroenteritis, Sjögren syndrome, scleroderma, and psoriasis.

11. The method of claim 8, wherein the malignant tumor disease is a solid cancer selected from the group consisting of lung cancer, gastric cancer, colorectal cancer, pancreatic cancer, ovarian cancer, uterine cancer, thyroid cancer, breast cancer, liver cancer, kidney cancer, prostate cancer, and mesothelioma, or any one of hematologic malignancy selected from the group consisting of leukemia, lymphoma, and multiple myeloma.

12. The method of claim 11, wherein the oxidized immunoglobulin has an anticancer immunotherapeutic effect by immunomodulation through activation of cytotoxic T cells.

13. The method of claim 1, further comprising an immunoadjuvant in addition to the oxidized immunoglobulin.

14. The method of claim 13, wherein the immunoadjuvant is one or more immunoadjuvants from the group consisting of aluminum hydroxide, calcium phosphate, tyrosine, monophosphoryl lipid A (MPL), or histamine.

15. A method of preventing or treating diseases associated with immune dysregulation, the method comprising:
isolating immunoglobulin from blood of a mammal itself or isolating immunoglobulin from blood of another mammal (step 1);
mixing and reacting the isolated immunoglobulin with an oxidant to prepare oxidized immunoglobulin (step 2); and
administering the oxidized immunoglobulin to an individual suffering from a disease associated with immune dysregulation (step 3).

16. The method of claim 15, wherein the oxidant is one or more oxidants from the group consisting of ozone (O₃), hydrogen peroxide (H₂O₂), and sodium hypochlorite (NaClO).

17. The method of claim 15, wherein the step 2 comprises mixing and reacting 0.1 to 4 μg of ozone per 1 mg of isolated immunoglobulin.

18. The method of claim 15, wherein the disease associated with immune dysregulation is any one from the group consisting of allergic diseases, chronic inflammatory diseases, autoimmune diseases, and malignant tumor diseases.

19. The method of claim 18, wherein the allergic disease is any one from the group consisting of bronchial asthma, allergic rhinitis, allergic conjunctivitis, atopic dermatitis, urticaria, food allergy, anaphylaxis, and drug allergy.

20. The method of claim 18, wherein the chronic inflammatory disease or autoimmune disease is any one from the group consisting of degenerative arthritis, chronic inflammatory gastroenteritis, ankylosing spondylitis, chronic pustular dermatitis, rheumatoid arthritis, systemic lupus erythematosus, pemphigus, autoimmune thyroiditis, autoimmune hepatitis, chronic inflammatory bowel disease, autoimmune nephritis, chronic inflammatory gastroenteritis, Sjögren syndrome, scleroderma, and psoriasis.

21. The method of claim 18, wherein the malignant tumor disease is a solid cancer selected from the group consisting of lung cancer, gastric cancer, colorectal cancer, pancreatic cancer, ovarian cancer, uterine cancer, thyroid cancer, breast cancer, liver cancer, kidney cancer, prostate cancer, and mesothelioma, or any one of hematologic malignancy selected from the group consisting of leukemia, lymphoma, and multiple myeloma.

22. A method of preparing a pharmaceutical composition for preventing or treating diseases associated with immune dysregulation, the method comprising:
isolating immunoglobulin from blood of a mammal itself or isolating immunoglobulin from blood of another mammal (step 1); and
mixing and reacting the isolated immunoglobulin with an oxidant to prepare oxidized immunoglobulins (step 2).

23. The method of claim 22, wherein the oxidant is one or more oxidants from the group consisting of ozone (O₃), hydrogen peroxide (H₂O₂), and sodium hypochlorite (NaClO).

24. The method of claim 22, wherein the step 2 comprises mixing and reacting 0.1 to 4 μg of ozone per 1 mg of isolated immunoglobulin.

25. The method of claim 22, wherein the disease associated with immune dysregulation is any one from the group consisting of allergic diseases, chronic inflammatory diseases, autoimmune diseases, and malignant tumor diseases.

26. The method of claim 25, wherein the allergic disease is any one from the group consisting of bronchial asthma, allergic rhinitis, allergic conjunctivitis, atopic dermatitis, urticaria, food allergy, anaphylaxis, and drug allergy.

27. The method of claim 25, wherein the chronic inflammatory disease or autoimmune disease is any one from the group consisting of degenerative arthritis, chronic inflammatory gastroenteritis, ankylosing spondylitis, chronic pustular dermatitis, rheumatoid arthritis, systemic lupus erythematosus, pemphigus, autoimmune thyroiditis, autoimmune hepatitis, chronic inflammatory bowel disease, autoimmune nephritis, chronic inflammatory gastroenteritis, Sjögren syndrome, scleroderma, and psoriasis.

28. The method of claim 25, wherein the malignant tumor disease is a solid cancer selected from the group consisting of lung cancer, gastric cancer, colorectal cancer, pancreatic cancer, ovarian cancer, uterine cancer, thyroid cancer, breast cancer, liver cancer, kidney cancer, prostate cancer, and mesothelioma, or any one of hematologic malignancy selected from the group consisting of leukemia, lymphoma, and multiple myeloma.
